(19) United States
(12) Patent Application Publication
(10) Pub. No.: US 2026/0289648 A1
(43) Pub. Date: Sep. 24, 2026

(54) SYSTEM

(71) Applicant: SoftBank Group Corp., Tokyo (JP)

(72) Inventor: Yota MASUDA, Tokyo (JP)

(73) Assignee: SoftBank Group Corp., Tokyo (JP)

(21) Appl. No.: 19/563,348

(22) Filed: Mar. 11, 2026

(30) Foreign Application Priority Data
Mar. 19, 2025 (JP) 2025-045004

(51) Int. Cl. G06Q 30/0601; G06N 3/045; G16H 20/60; G16H 50/30

(52) U.S. Cl. CPC G06Q 30/06311; G06N 3/045; G06Q 30/06313; G16H 20/60; G16H 50/30

(57) ABSTRACT

A system includes a processor that is configured to receive information from a user, analyze the received information and, using a generative artificial intelligence model, generate a cooking method according to at least one of a price, a nutritional value, and an inventory status, and analyze a purchase history and an emotional state of the user and, based on a result of the analysis, propose a cooking method suitable for at least one of a specific nutritional value, a budget, and an emotion.

### CROSS-REFERENCE TO RELATED APPLICATION

This application is based on and claims priority under 35 USC 119 from Japanese Patent Application No. 2025-045004 filed on Mar. 19, 2025, the disclosure of which is incorporated by reference herein.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

Conventional recipe recommendation systems generally rely on static rule-based logic, fixed databases of recipes, and simple user preference filters such as cuisine type or dietary restrictions. Such systems are not capable of flexibly generating cooking methods that dynamically reflect real-time factors including item price fluctuations, detailed nutritional values, and inventory status at nearby retailers or in a user's household. As a result, users cannot easily obtain cooking methods that are optimized for their current budget, available ingredients, or desired nutritional balance.

Furthermore, conventional systems typically do not take into account a user's emotional state or fine-grained history of purchases when proposing cooking methods. Even where past purchase information is used, it is usually limited to recommending items that the user has bought before, without considering how those items can be combined into cooking methods that align with the user's current emotional condition, such as stress, fatigue, or a desire for comfort food. Consequently, existing solutions fall short of providing highly personalized proposals that adapt to both quantitative factors (price, nutrition, inventory) and qualitative factors (user emotion, mood).

Additionally, traditional systems often require the user to manually perform multiple steps, such as checking supermarket flyers, comparing prices, looking up nutritional data, and searching for suitable recipes separately. This increases the cognitive load on the user and makes it difficult to quickly decide on an appropriate cooking method that satisfies constraints on budget, nutritional value, and emotional preference. Therefore, there is a need for a system that can automatically and comprehensively analyze user-provided information, purchase history, emotional state, and product information of nearby retailers, and generate or propose cooking methods that simultaneously consider price, nutritional value, inventory status, and user emotion by using a generative artificial intelligence model.

### SUMMARY

In order to solve at least part of the above-described problems, according to one aspect of the present invention, there is provided a system comprising a processor, wherein the processor is configured to receive information from a user, analyze the received information, and, using a generative artificial intelligence model, generate a cooking method according to at least one of a price, a nutritional value, and an inventory status. The processor is further configured to analyze a purchase history and an emotional state of the user and, based on a result of the analysis, propose a cooking method suitable for at least one of a specific nutritional value, a budget, and an emotion.

In one embodiment, the processor is configured to receive a prompt from the user and, in a case where the prompt is for instructing a proposal of a cooking method, propose the cooking method using the generative artificial intelligence model. By interpreting such prompts, the processor can flexibly respond to various user requests, including requests specifying a desired calorie range, a cost per person, or a particular emotional condition, and can generate new or customized cooking methods that satisfy those constraints.

In another embodiment, the processor is configured to refer to product information of nearby retailers and, using the generative artificial intelligence model, propose a cooking method using ingredients that are purchasable within a specific budget. The processor may acquire or update product information including price, nutritional value, and inventory status from one or more external servers or databases associated with nearby supermarkets or retailers. Based on this information, the processor can select ingredients that satisfy a user-specified budget and nutritional target, and then cause the generative artificial intelligence model to generate detailed cooking methods utilizing those ingredients.

By integrating the analysis of user purchase history, emotional state, and retailer product information, as well as employing a generative artificial intelligence model for recipe generation, the system according to the present invention can provide the user with cooking methods that are dynamically optimized for real-time price and inventory conditions, personalized nutritional requirements, and the user's current emotional condition, thereby reducing the burden on the user and improving the relevance and usefulness of proposed cooking methods.

The term “system” refers to an arrangement of at least one hardware device, software component, or a combination thereof, configured to execute the functions described in the present specification and claims, including processing, storage, communication, and user interaction.

The term “processor” refers to any hardware or virtual computing element capable of executing instructions, including but not limited to a central processing unit (CPU), a graphics processing unit (GPU), a microcontroller, a digital signal processor, a programmable logic device, or a cloud-based computing resource, and may be implemented as a single unit or as a plurality of distributed units.

The term “user” refers to a human individual who interacts, directly or indirectly, with the system by providing information, prompts, preferences, or other inputs, and who receives cooking methods or proposals generated by the system.

The term “information from a user” refers to any data provided by the user, explicitly or implicitly, including prompts, text input, voice input, selections from a graphical user interface, historical usage data, preferences, constraints on price or nutritional value, and indications of an emotional state.

The term “prompt” refers to an instruction, query, request, or other input expressed by the user in natural language or structured form, which indicates, directly or indirectly, that the system is to generate, modify, or propose a cooking method.

The term “analyze” refers to any process in which the processor interprets, classifies, transforms, extracts features from, or otherwise processes data using deterministic algorithms, statistical methods, machine learning models, or any combination thereof.

The term “generative artificial intelligence model” refers to a machine learning model capable of generating new content, including text representing cooking methods or recipes, based on input data and learned parameters, and includes, for example, generative neural networks, transformer-based language models, variational autoencoders, generative adversarial networks, or any other generative model architecture.

The term “cooking method” refers to any representation of a way of preparing food, including, but not limited to, a recipe, a list of ingredients, ingredient quantities, preparation steps, cooking times, temperatures, and serving suggestions, and may be expressed in textual, symbolic, or structured data form.

The term “price” refers to a monetary amount associated with purchasing an ingredient, a set of ingredients, or a ready-made product, and may include unit prices, total prices for a package, discounted prices, or dynamic prices provided by retailers.

The term “nutritional value” refers to any information related to the nutritional composition of ingredients or meals, including, but not limited to, energy (calories), macronutrients (proteins, fats, carbohydrates), micronutrients (vitamins, minerals), or any other nutritional indicator relevant to dietary considerations.

The term “inventory status” refers to information indicating the availability, stock level, or stock-out condition of ingredients or products, either in a retailer's inventory or, if available, in a user's home inventory, and may be represented in quantitative or qualitative form.

The term “purchase history” refers to records of past purchases associated with the user, including information such as product names, quantities, purchase dates, prices, and retailers, which may be obtained from external systems, user accounts, receipts, or manual input.

The term “emotional state” refers to any representation of the user's current or recent emotional condition, mood, or affect, such as stress, fatigue, happiness, sadness, or desire for comfort, which may be inferred from user input, behavioral data, biometric signals, or explicit self-reporting by the user.

The term “specific nutritional value” refers to one or more target conditions or constraints related to nutrition, such as a maximum or minimum calorie amount, a target macronutrient ratio, a dietary restriction, or a particular nutritional goal specified or implied by the user.

The term “budget” refers to a monetary constraint or range specified or inferred for purchasing ingredients or preparing a meal, including a total allowable amount, a per-person amount, or a per-meal amount, used by the processor to filter or generate cooking methods.

The term “emotion” refers to a qualitative aspect of the user's internal state or mood, including types or categories of emotional states such as comfort-seeking, celebratory, relaxing, or energizing, as used for tailoring proposed cooking methods to the user's psychological preferences.

The term “nearby retailers” refers to one or more physical or online stores that are geographically or logically associated with the user, from which the user can reasonably obtain ingredients, and which provide, or are associated with, product information accessible to the system.

The term “product information” refers to data describing items offered by retailers, including but not limited to product names, categories, prices, package sizes, nutritional values, and inventory status, as well as any metadata used to identify or classify the products.

The term “ingredients that are purchasable within a specific budget” refers to one or more food items whose combined price does not exceed a budget amount set or inferred by the system, taking into account unit prices, required quantities, and any applicable discounts or promotions.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

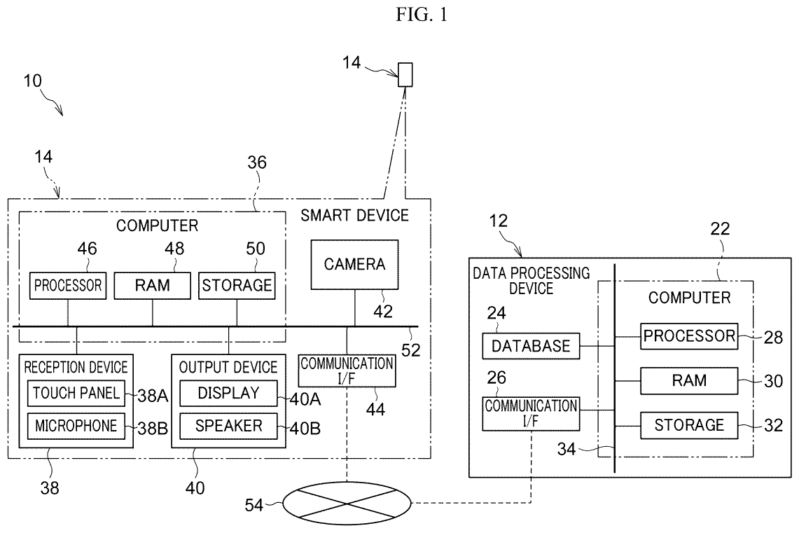

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

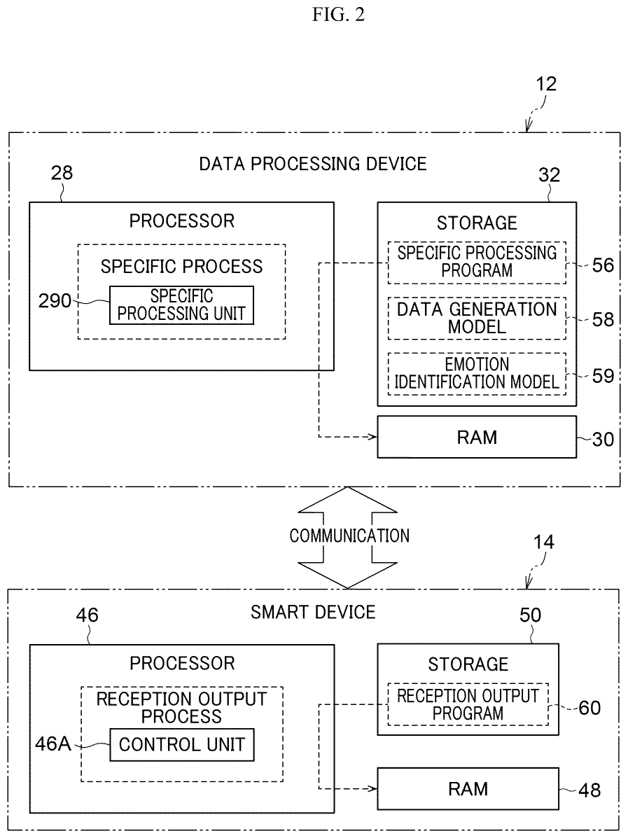

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventional recipe recommendation systems and shopping support systems rely mainly on fixed rule sets or simple matching between pre-stored recipes and user preferences. Such systems typically handle user inputs, ingredient lists, and store product information as separate, loosely coupled data streams. As a result, these systems suffer from several technical limitations in how a computer processes, structures, and utilizes heterogeneous data to generate cooking instructions.

First, conventional systems do not efficiently integrate multiple types of data sources—such as local purchase history stored on a user device, dynamic merchandise information from multiple commercial facilities, and free-form natural language requests—into a unified, machine-usable representation. In many cases, the system simply passes the raw user query to a remote model or search engine without systematically encoding structured context, such as normalized quantities, nutritional values, or budget constraints. This causes suboptimal utilization of available data and leads to increased computational overhead on downstream processing components, because the downstream components must infer or reconstruct structure from noisy text.

Second, when generative AI models are used, existing approaches often treat the model as a black box and provide only a short, unstructured prompt sentence. Without a precise, programmatically constructed extended prompt that includes normalized ingredient data, budget-calculated product sets, and clearly defined output formats, a generative AI model may generate responses that violate budget or nutritional constraints, omit necessary details, or require repeated user interactions to correct errors. This lack of structured prompt engineering at the system level results in inefficient use of computing resources and increased latency, since more inferences and post-processing are needed to reach an acceptable result.

Third, traditional systems do not adequately perform pre-processing on the user device or server to reconcile and normalize units, sum quantities, and compute nutritional metrics before invoking the generative AI model. Instead, any such processing is often manual, ad hoc, or pushed into the model itself. This places unnecessary computational burden on the generative AI model, reduces determinism in the output, and wastes bandwidth because the same raw data may be transmitted repeatedly without being distilled into an efficient, structured context.

Fourth, conventional solutions do not provide a systematic mechanism by which the processor constructs an extended prompt that explicitly encodes constraint conditions, such as budget, intake calories, serving number, and ingredient usage, together with dynamically extracted combinations of purchasable items within a budget from nearby commercial facilities. Without such a mechanism, the computer system cannot reliably direct the generative AI model to perform constrained generation in a way that is verifiable and traceable from the system's intermediate data structures, thereby limiting the technical robustness and predictability of the overall pipeline.

Fifth, many existing systems do not generate a structured internal representation of generative outputs. They often treat the generated text as a monolithic block and only display it directly to the user. This approach prevents efficient downstream processing, such as segregating owned ingredients from to-be-purchased ingredients, generating shopping lists, or reusing structured fields (e.g., estimated total cost, total calories) in other components. Consequently, the computer's ability to reuse and recombine results is impaired, leading to redundancy, higher memory usage, and reduced scalability of the system.

Therefore, there is a need for a computer-implemented system and method that improves how a processor: (i) acquires, normalizes, and integrates user purchase history and external merchandise data, (ii) programmatically constructs an extended prompt sentence embedding structured context and constraint conditions for a generative AI model, (iii) invokes the model in a manner that is computationally efficient and constrained by explicit parameters, and (iv) converts the generated text into structured output for subsequent interface rendering and data reuse. Such a solution should improve the technical functioning of the overall computer system by reducing unnecessary computation at the inference stage, lowering network bandwidth usage through more compact and meaningful prompts, and enabling more deterministic and verifiable generation of recipe-related outputs.

The specific processing by the specific processing unit 290 of the data processing device 12 in Example 1 is realized by the following means.

The present invention provides a server comprising a processor configured to cooperate with an application program executed on an information processing apparatus, the processor being configured to receive, via the application program, a prompt sentence input by a user; to cause the application program to extract ingredient information from purchase history data stored in a storage device of the information processing apparatus and to convert the ingredient information into structured data including attributes related to nutritional values and quantities; to cause the application program to acquire, via a communication network, merchandise information of commercial facilities including product attribute data such as price information, stock information, and nutritional information, and to associate the ingredient information with the product attribute data on the basis of product identification information or name information; to cause the application program to perform unit normalization, quantity aggregation, and nutritional value calculation on the ingredient information and the product attribute data to generate basic data for specifying usable ingredients and candidate products to be purchased; to cause the application program to analyze the prompt sentence on the basis of predetermined rules or natural language processing to extract constraint conditions including at least one of a budget condition, a nutritional condition, an intake calorie condition, a serving number condition, and an ingredient usage condition; to cause the application program to construct context information including the constraint conditions, the ingredient information, and the product attribute data as an extended prompt sentence in a hierarchical or segmented text format for input to a generative AI model, and to include specifications of an output format in the extended prompt sentence; to input the extended prompt sentence to the generative AI model and, based on inference processing by the generative AI model, generate one or more cooking method candidates expressed in natural language that satisfy the constraint conditions; to analyze an output from the generative AI model and convert the one or more cooking method candidates into structured results including at least a dish name, a list of ingredients, quantities, cooking steps, an estimated total cost, and an estimated total calorie amount; and to cause the application program to generate a user interface that, on the basis of the structured results, distinguishes between ingredients possessed by the user and ingredients to be purchased and presents the cooking steps and indicators related to the constraint conditions to the user. This enables the computer system to improve internal data handling and inference efficiency by performing structured pre-processing and constraint extraction before invoking the generative AI model, to reduce processing load and bandwidth usage through a compact extended prompt that encodes normalized ingredient and merchandise data, and to enhance the determinism and reusability of generated content by converting model outputs into structured representations that drive dynamic user interfaces and downstream computational functions.

The term “processor” refers to a hardware or virtual computation unit, such as a central processing unit or an execution core, that executes instructions of a program to perform arithmetic operations, logical operations, control operations, and data transfer operations.

The term “information processing apparatus” refers to an electronic device, such as a terminal, mobile device, or personal computer, that includes at least a processor, a memory, and a communication interface, and that executes an application program to process data.

The term “application program” refers to software executed by the information processing apparatus that provides a user interface, acquires and processes data including purchase history and merchandise information, constructs prompt sentences, and communicates with a server or a generative AI model.

The term “prompt sentence” refers to a text string input by a user or generated by the application program that expresses an instruction, request, or condition to be processed by the system or a generative AI model.

The term “extended prompt sentence” refers to a prompt sentence that has been programmatically constructed or augmented by the application program or the processor to include structured context information, such as constraint conditions, ingredient information, product attribute data, and output format specifications, in a hierarchical or segmented text format.

The term “purchase history data” refers to data records stored in a storage device of the information processing apparatus or a server, each record describing at least an item that has been purchased, a quantity, a purchase date, and optionally an expiration date or category information.

The term “ingredient information” refers to data derived from purchase history data or other sources that represents food materials possessed by the user, including at least an item identifier, an item name, a quantity, and optionally nutritional values and unit information.

The term “merchandise information” refers to data provided by or on behalf of commercial facilities that describes products available for sale, including product identifiers, names, prices, stock statuses, nutritional values, and related attributes.

The term “product attribute data” refers to structured data representing attributes of a product included in merchandise information, such as price information, stock information, nutritional information, and identification information.

The term “storage device” refers to a physical or logical data storage component, such as a memory device, a database, or a non-volatile storage medium, that stores purchase history data, ingredient information, or other data used by the system.

The term “communication network” refers to any wired or wireless network infrastructure that enables data exchange between the information processing apparatus, the server, and external data sources, including public networks and private networks.

The term “generative AI model” refers to a trained artificial intelligence model, such as a neural network or transformer-based model, that receives a prompt sentence and generates natural language text or other data as an output in accordance with learned patterns and the provided context.

The term “constraint conditions” refers to one or more conditions extracted from a prompt sentence or specified by the system, including at least a budget condition, a nutritional condition, an intake calorie condition, a serving number condition, or an ingredient usage condition, which the generative AI model is required or requested to satisfy when generating a cooking method.

The term “budget condition” refers to a constraint condition that specifies an upper limit or range for monetary cost related to ingredients or recipes.

The term “nutritional condition” refers to a constraint condition that specifies one or more requirements related to nutritional values, such as protein amount, fat amount, or specific nutrient limits or targets.

The term “intake calorie condition” refers to a constraint condition that specifies an upper limit, lower limit, or target value for caloric intake per serving, per person, or per meal.

The term “serving number condition” refers to a constraint condition that specifies a number of servings or persons for which a cooking method or recipe is to be designed.

The term “ingredient usage condition” refers to a constraint condition that specifies preferences or requirements regarding the use of certain ingredients, such as mandatory use of specific items, avoidance of specific items, or prioritization of ingredients already possessed by the user.

The term “basic data” refers to processed data generated from ingredient information and product attribute data, including normalized units, aggregated quantities, and calculated nutritional values, which is used as a basis for specifying usable ingredients and candidate products to be purchased.

The term “unit normalization” refers to a data processing operation that converts quantities expressed in different measurement units into a consistent unit system to enable aggregation and comparison.

The term “quantity aggregation” refers to an operation that calculates a total quantity of an ingredient or product by summing multiple records or entries that refer to the same or equivalent item.

The term “nutritional value calculation” refers to an operation that computes nutritional metrics, such as total calories, protein, fat, or carbohydrates, based on quantities and per-unit nutrition data of ingredients or products.

The term “context information” refers to a collection of data elements, including constraint conditions, ingredient information, and product attribute data, which is assembled into a structured or semi-structured format and provided to the generative AI model as part of an extended prompt sentence.

The term “output format specifications” refers to explicit instructions included in the extended prompt sentence that define the desired structure, sections, or fields of the generative AI model's output, such as titles, ingredient lists, steps, and numerical estimates.

The term “cooking method candidate” refers to a proposed recipe or cooking procedure generated by the generative AI model in response to an extended prompt sentence, including at least a description of a dish and steps to prepare it.

The term “structured results” refers to data obtained by parsing and organizing a cooking method candidate into defined fields, such as dish name, list of ingredients, quantities, cooking steps, estimated total cost, and estimated total calorie amount.

The term “dish name” refers to a text label that identifies a cooking method candidate as a particular meal, recipe, or prepared food item.

The term “list of ingredients” refers to a structured enumeration of ingredients required for a cooking method candidate, including at least ingredient names and associated quantities.

The term “cooking steps” refers to an ordered sequence of instructions describing how to prepare a dish using the listed ingredients.

The term “estimated total cost” refers to a calculated or inferred monetary amount associated with the ingredients required for a cooking method candidate, based on product attribute data such as price information.

The term “estimated total calorie amount” refers to a calculated or inferred total caloric value associated with a cooking method candidate, based on nutritional information and ingredient quantities.

The term “user interface” refers to a visual, auditory, or interactive presentation generated by the application program on the information processing apparatus, through which the user can view structured results, distinguish between owned ingredients and ingredients to be purchased, and interact with the system.

The term “ingredients possessed by the user” refers to ingredients that are inferred or determined, from purchase history data or stored stock information, to be currently available to the user for cooking.

The term “ingredients to be purchased” refers to ingredients that are not currently available to the user, but are identified from merchandise information as candidates for purchase in order to complete a cooking method candidate.

The term “commercial facility” refers to a physical or virtual retail or distribution entity, such as a store or marketplace, that offers products or ingredients for sale and for which merchandise information is available.

In one embodiment, the invention is implemented by cooperation between a server, a terminal, and a user. The terminal corresponds to an information processing apparatus, such as a smartphone, tablet, or personal computer, that executes an application program. The server corresponds to a remote computing system that executes a generative AI model and provides inference results to the terminal over a communication network.

The terminal includes at least a central processing unit (CPU), a main memory, a non-volatile storage device such as flash memory, a display device such as a liquid crystal display or organic EL display, an input interface such as a touch panel or keyboard, and a communication interface such as a wireless communication module (for example, Wi-Fi or a cellular communication module). The terminal executes an operating system such as a mobile operating system or a desktop operating system. The application program runs on this operating system and uses a local database, for example an embedded relational database such as SQLite or a platform-specific storage mechanism such as a key-value store or an object-relational mapping layer (for example, Room or Core Data), to store purchase history data and local configuration data.

The server includes one or more processors, a main memory, a non-volatile storage system such as a magnetic disk device or solid-state drive, and a network interface. In one embodiment, the server includes one or more accelerator devices such as graphics processing units (GPUs), tensor processing units (TPUs), or other specialized computation devices. The server executes a server-side operating system and an application framework such as a web application framework. The server hosts a generative AI model implemented by a neural network architecture, such as a transformer-based language model having multiple self-attention layers, feed-forward layers, and layer normalization components.

The user operates the terminal by launching the application program and entering a prompt sentence. The prompt sentence is a text string, for example, “I want to cook dinner around 600 kcal with ingredients I already have at home.” or “I want to cook dinner for one person with a budget of 250 yen at a nearby supermarket.” The terminal displays a text input area on the screen and receives the characters through the input interface. The terminal stores the prompt sentence in the main memory and, optionally, in temporary storage.

The terminal maintains purchase history data in the local database. The purchase history data includes, for each purchased item, at least an item identifier, a category, a quantity, a unit, a purchase date, and optionally an expiration date and basic nutritional information. In one embodiment, the terminal stores this data in a table having fields such as item_id, item_name, quantity_value, quantity_unit, purchase_date, expiration_date, and category_code. The terminal may also store inferred stock levels by subtracting estimated usage when the user selects and confirms a recipe.

The terminal acquires merchandise information from external data sources through the communication network. The merchandise information describes products sold in commercial facilities, and includes attributes such as product_id, product_name, price_value, price_currency, stock_status, and nutritional values (for example, calories, protein, fat, carbohydrates). The terminal receives this merchandise information as structured data, for example in a text-based serialized format via a web application programming interface, and stores the parsed data in memory structures such as arrays, lists, or maps.

The terminal performs specific data processing operations on the purchase history data and the merchandise information. The terminal performs unit normalization by converting quantities expressed in different units to common canonical units. For example, the terminal converts “1 pack of chicken (300 g)” and “200 g of chicken” into a single canonical unit of grams, and aggregates the total available amount of chicken. The terminal uses conversion rules stored in the local database or in configuration files. This processing allows the terminal to generate consistent feature vectors for downstream computation, and reduces the risk of numerical errors when the generative AI model reasons about available quantities.

The terminal performs quantity aggregation by summing quantities of identical or equivalent items across multiple purchase records. The terminal matches items by product identifiers or, when identifiers are not available, by applying string normalization and similarity measures to item names and categories. By aggregating quantities, the terminal reduces the dimensionality of the ingredient representation and compresses multiple records into a unified stock representation. This reduces the size of the data to be transmitted to the server and decreases the computational cost of parsing the context on the server.

The terminal performs nutritional value calculation based on available nutritional reference data. The terminal multiplies per-unit nutritional values (for example, calories per 100 g) by the normalized quantities to compute total nutritional contributions of each ingredient held by the user. The terminal may also compute derived metrics such as macro-nutrient ratios or approximate glycemic load. These calculations transform raw purchase records into structured nutritional feature vectors that are directly usable by the generative AI model or by auxiliary processing modules. Because the terminal executes these computations locally, the server receives pre-computed, normalized nutritional vectors, which improves overall system performance.

The terminal analyzes the prompt sentence using predetermined rules or natural language processing techniques. In one embodiment, the terminal performs keyword extraction and pattern matching to detect expressions of constraints such as “600 kcal”, “under 250 yen”, “for one person”, or “low salt”. The terminal maps these expressions to structured constraint fields, such as a maximum calorie limit, a budget condition, a serving number condition, and an ingredient usage condition. In another embodiment, the terminal uses a lightweight natural language processing module, such as a smaller classification model or rule-based parser, to tag entities and extract constraint values. This local extraction avoids sending unnecessary free-form user text to the server and improves privacy.

The terminal constructs an extended prompt sentence for the generative AI model. The terminal assembles the extracted constraints, the normalized ingredient data, and the selected merchandise information into a hierarchical or segmented text format. For example, the terminal concatenates a “User request” section, a “Constraints” section, an “Available ingredients” section, a “Nearby products” section, and an “Output format” section. The extended prompt sentence may be, for example:

“User request: I want to cook dinner around 600 kcal with ingredients I already have at home.

### Constraints:

Maximum 600 kcal per serving
Number of servings: 2
Prioritize using ingredients already possessed by the user

### Available Ingredients:

300 g chicken breast
2 carrots
1 broccoli
200 g tofu
500 g rice

### Output Format:

1. Recipe title
2. Ingredient list with quantities
3. Step-by-step cooking instructions
4. Approximate calories per serving
5. If additional store items are needed, list them with price and store name.”

In another example, the extended prompt sentence is:

“User request: I want to cook dinner for one person with a budget of 250 yen at a nearby supermarket.

### Constraints:

Maximum 250 yen total ingredient cost
Number of servings: 1

### Nearby Store Products:

Cabbage (¼ head), 80 yen, about 50 kcal, in stock
Egg (1 piece), 30 yen, about 80 kcal, in stock
Rice (200 g), 70 yen, about 300 kcal, in stock
Tofu (150 g), 60 yen, about 90 kcal, out of stock

### Output Format:

1. Recipe title
2. Ingredient list with prices
3. Total cost
4. Cooking steps.”

By constructing the extended prompt sentence in this structured manner, the terminal compresses the relevant context into a concise, machine-optimized representation. The generative AI model receives a prompt that already encodes numeric constraints, normalized ingredient information, and explicit output structure. As a result, the generative AI model performs fewer internal inference steps to interpret the task constraints, and the server reduces the need for post-hoc corrections or re-queries.

The server receives the extended prompt sentence, tokenizes the text according to the tokenizer defined for the generative AI model, and maps tokens to numerical indices. The server constructs input tensors containing token identifiers, positional encodings, and segment identifiers, and feeds these tensors into the generative AI model. In one embodiment, the generative AI model is a transformer-based language model including multiple layers of multi-head self-attention and feed-forward networks. Each self-attention layer uses query, key, and value matrices to compute attention weights over prior tokens. The server stores the parameters of the generative AI model—such as weight matrices and bias vectors—in the main memory or on the accelerator device.

The server executes the generative AI model by performing matrix multiplications, nonlinear activation functions such as rectified linear units or gating functions, and normalization operations. The server applies a decoding strategy such as greedy decoding, beam search, or sampling with parameters such as temperature, top-k, or nucleus sampling. The server generates tokens iteratively until a termination condition is met, such as generating an end-of-sequence marker or reaching a predetermined maximum token length. The server reconstructs the generated text from the tokens.

In one embodiment, the generative AI model is trained in advance using a training dataset that includes recipe texts, ingredient lists, nutritional information, and example prompts. The server trains the model using a loss function such as cross-entropy between predicted token distributions and ground-truth tokens. The server performs gradient-based optimization, such as stochastic gradient descent or adaptive methods, and updates the model parameters by backpropagation. The server may perform data augmentation by rephrasing prompt sentences, scaling numeric values within certain ranges, or permuting sections to increase model robustness. By training on structured recipe data with explicit constraints, the generative AI model learns to respect numeric constraints and to output specific fields, such as total calories and costs, in a predictable format.

The server optionally fine-tunes the generative AI model on usage logs generated by the system. When the terminal and the server store accepted recipes along with constraint conditions and structured outputs, the server may use these logs to refine the model so that it more accurately produces recipes that conform to user constraints and system-specified structure. This continuous fine-tuning further improves accuracy and reduces the number of inference iterations required to obtain an acceptable output.

The server analyzes the generated text to detect sections corresponding to the recipe title, ingredient list, cooking steps, and numeric values. In one embodiment, the generative AI model itself includes explicit markers or section headers, and the server parses the text by searching for those markers. In another embodiment, the server applies simple pattern matching or regular expressions to extract numeric values such as “Total cost: 230 yen” or “Total calories per serving: 580 kcal.” The server converts these values into numerical data types and stores them in structured data objects. This structured representation allows the server and the terminal to perform further computations, such as recomputing per-person costs or checking whether the generated recipe satisfies the original constraints.

The terminal receives the structured results from the server, stores them in local memory, and displays a user interface. The terminal distinguishes between ingredients that the user already possesses and ingredients that must be purchased. For example, the terminal may display two sections: “Use ingredients at home” and “Buy at store.” This separation is achieved algorithmically by matching ingredient names returned from the structured results with the normalized ingredient list derived from the purchase history and the merchandise information. The terminal can use this structured mapping to generate shopping lists, to suggest substitution options, or to update local stock levels. This structured internal handling goes beyond merely displaying text to the user and improves data management and reuse.

The system improves computer technology in several ways. Because the terminal performs data normalization, aggregation, and constraint extraction prior to sending the extended prompt sentence to the server, the system reduces the volume of raw data transmitted over the network and lowers the computational load on the server-side inference pipeline. The generative AI model operates on a compact, feature-rich prompt, which reduces ambiguity and accelerates convergence to a valid output. The use of structured, extended prompts and structured parsing of outputs enables deterministic behavior, simplifies validation, and facilitates caching or re-use of intermediate computations. The system thereby improves processing speed, accuracy of constraint satisfaction, data management efficiency, and overall computational efficiency.

The system uses non-conventional processing flows compared to typical human workflows.

A human user would ordinarily read purchase records, inspect store flyers, and manually calculate total costs and calories before choosing a recipe. In contrast, the terminal and the server automatically transform heterogeneous data into unified numeric and symbolic representations, apply a trained neural architecture with learned attention patterns over these structured inputs, and generate constrained natural language outputs using explicit algorithmic decoding strategies. The unit normalization rules, constraint extraction procedures, extended prompt construction logic, and structured output parsing collectively form a technical pipeline that is specific to the computer implementation and not merely an automation of human reasoning.

In alternative embodiments, the terminal may execute a smaller generative AI model locally for certain tasks, such as summarizing purchase history or suggesting minor recipe variations, while the server executes a larger generative AI model for full recipe generation. In another embodiment, the server may delegate certain pre-processing tasks to a dedicated microservice that performs advanced nutritional computations or ingredient clustering using statistical algorithms or graph-based methods. In yet another embodiment, the system may adjust the extended prompt sentence format dynamically based on device capabilities or network conditions to further reduce communication overhead and to optimize latency.

In all embodiments, the server and the terminal cooperate to transform raw user input and heterogeneous data into a constrained, structured representation, to invoke a generative AI model that is architected and trained to respect these constraints, and to present the results in a form that enables further machine-level processing and user interaction. Through this arrangement, the system improves not only the convenience of recipe selection but also the technical functioning of the underlying computing system in terms of data handling, computational efficiency, and output reliability.

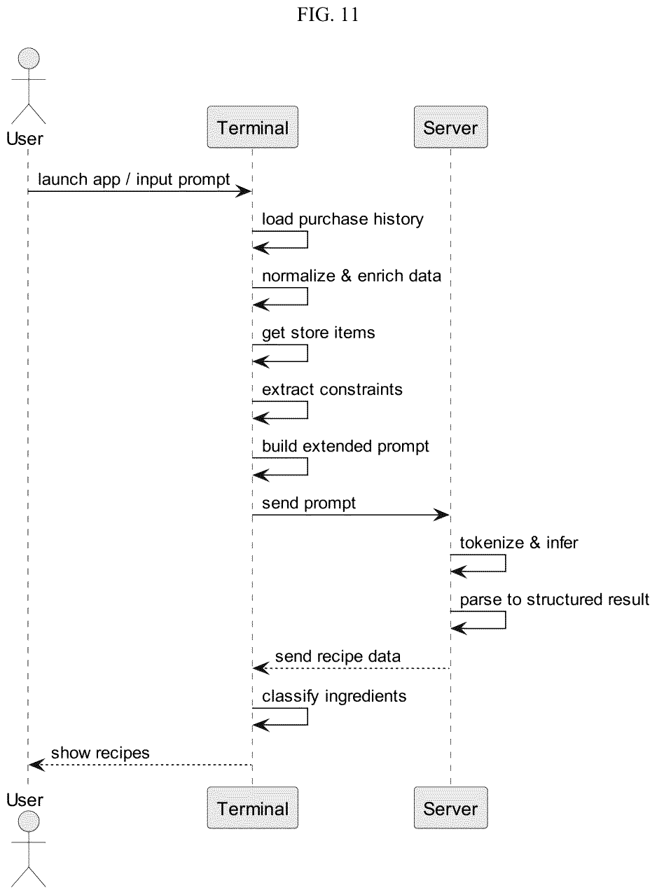

The following describes the processing flow using FIG. 11.

### Step 1:

The user operates the terminal to launch the application program and to input a prompt sentence.

The input of Step 1 is a raw text string entered by the user, such as “I want to cook dinner around 600 kcal with ingredients I already have at home.” or “I want to cook dinner for one person with a budget of 250 yen at a nearby supermarket.”

The terminal displays a text input field on the screen, receives keystrokes or touch input through the input interface, and stores the completed prompt sentence in main memory as a character string.

The output of Step 1 is a stored prompt sentence associated with a current session identifier inside the terminal.

### Step 2:

The terminal accesses a local storage device to retrieve purchase history data corresponding to the user.

The input of Step 2 is a user identifier or profile key and the prompt sentence from Step 1.

The terminal opens a connection to a local database, executes a query to select purchase records for the user, and loads fields such as item identifier, item name, quantity, unit, purchase date, and expiration date into in-memory data structures.

The terminal filters out expired items or irrelevant categories based on comparison of expiration dates with the current system time and category codes with predefined configuration rules.

The output of Step 2 is an in-memory list or table of valid purchase history entries that represent candidate ingredients possessed by the user.

### Step 3:

The terminal aggregates quantities and normalizes units for the purchase history data.

The input of Step 3 is the list of valid purchase history entries from Step 2.

The terminal groups entries by a common key, such as item identifier or a normalized item name, and sums quantity values for each group to compute total available quantities.

The terminal converts heterogeneous quantity units (for example, pieces, grams, milliliters) into canonical units based on predefined conversion tables stored in the local database or configuration files.

The terminal updates each grouped entry with a normalized quantity and unit, replacing the original raw quantities.

The output of Step 3 is a normalized ingredient list that describes, for each ingredient, a unique identifier, a normalized name, and a total normalized quantity.

### Step 4:

The terminal acquires merchandise information from commercial facilities via a communication network.

The input of Step 4 is the user's location information or region setting and configuration data identifying one or more commercial facilities or external product information providers.

The terminal uses a location service API to obtain the current geographic coordinates or a predefined region code, constructs network requests including the location data, and sends these requests to external servers that provide merchandise information.

The terminal receives responses containing product attribute data, parses the responses to extract fields such as product identifier, product name, price, stock status, and nutritional values, and stores this information in in-memory collections indexed by product identifier or store identifier.

The output of Step 4 is a merchandise information dataset for nearby or relevant commercial facilities, represented as structured product attribute records.

### Step 5:

The terminal associates the normalized ingredient list with the merchandise information and computes nutritional values for possessed ingredients.

The input of Step 5 is the normalized ingredient list from Step 3 and the merchandise information dataset from Step 4.

The terminal matches each normalized ingredient with corresponding products in the merchandise information by comparing identifiers, names, and categories, and builds linkages between ingredient records and product attribute records.

The terminal reads nutritional values from matching merchandise entries or from a local nutritional reference table, multiplies per-unit nutritional values by normalized quantities for each ingredient, and computes total nutritional metrics such as total calories, protein, fat, and carbohydrates per ingredient.

The terminal stores these computed nutritional values as additional fields in the ingredient records.

The output of Step 5 is an enriched ingredient dataset that includes, for each ingredient, normalized quantities and calculated nutritional metrics, and a mapping between possessed ingredients and purchasable products.

### Step 6:

The terminal analyzes the prompt sentence to extract constraint conditions.

The input of Step 6 is the prompt sentence from Step 1 and configuration data describing patterns or rules for constraint extraction.

The terminal applies rule-based parsing or a lightweight natural language processing module to detect expressions related to budget, calories, number of servings, nutritional preferences, and ingredient usage requirements.

The terminal converts detected expressions into structured constraint fields, such as a maximum budget amount, a maximum calorie per serving, a required serving count, and flags for prioritizing owned ingredients or avoiding specific ingredients.

The terminal stores these constraint conditions in memory as a structured constraint object associated with the session.

The output of Step 6 is a structured constraint object that numerically and symbolically encodes the conditions expressed by the user in the prompt sentence.

### Step 7:

The terminal selects purchasable ingredient combinations from merchandise information under the extracted budget condition.

The input of Step 7 is the merchandise information dataset from Step 4 and the structured constraint object from Step 6.

The terminal filters the merchandise information to products available at commercial facilities within the user's region and in stock, and then computes cost combinations that satisfy the budget condition by summing price values of candidate sets of products.

The terminal may apply heuristic or algorithmic selection methods, such as greedy selection by descending nutritional density or dynamic programming over price and nutritional values, to generate candidate sets of purchasable ingredients that fit within the budget.

The terminal stores selected combinations along with their total cost and approximate nutritional values for inclusion in the context for the generative AI model.

The output of Step 7 is a set of purchasable ingredient combinations, each annotated with its total cost and derived nutritional metrics.

### Step 8:

The terminal constructs an extended prompt sentence for the generative AI model.

The input of Step 8 is the prompt sentence from Step 1, the enriched ingredient dataset from Step 5, the structured constraint object from Step 6, and the purchasable ingredient combinations from Step 7.

The terminal formats these inputs into a hierarchical or segmented text structure that contains explicit sections such as “User request,” “Constraints,” “Owned ingredients,” “Purchasable ingredients within budget,” and “Output format requirements.”

The terminal converts numeric fields, such as cost and calories, into textual descriptions with consistent units, and includes explicit instructions specifying that the generative AI model should output a recipe title, a list of ingredients with quantities, step-by-step instructions, and estimated total cost and calories.

The terminal concatenates all sections into a single extended prompt sentence string, ensuring that separators and headings are inserted to enable reliable parsing by the generative AI model.

The output of Step 8 is a fully constructed extended prompt sentence that encapsulates all relevant structured context for recipe generation.

### Step 9:

The terminal transmits the extended prompt sentence to the server and awaits a response.

The input of Step 9 is the extended prompt sentence from Step 8 and communication configuration information such as server endpoint addresses and authentication tokens.

The terminal packages the extended prompt sentence and related metadata into a request message, sends the request to the server via a secure communication protocol, and sets up a session or correlation identifier to match the incoming response.

The terminal temporarily suspends further processing related to recipe generation and may display a loading indicator until the server returns results.

The output of Step 9 is a network request delivered to the server that contains the extended prompt sentence and associated context.

### Step 10:

The server receives the extended prompt sentence and prepares input tensors for the generative AI model.

The input of Step 10 is the network request containing the extended prompt sentence from Step 9.

The server validates the request, extracts the extended prompt sentence, and applies a tokenizer specific to the generative AI model to convert the text into a sequence of token identifiers.

The server constructs input tensors that encode the token identifiers and positional indices, and, if applicable, segment information distinguishing sections of the extended prompt.

The server allocates memory on a computation device such as a GPU or CPU and loads the input tensors into this memory.

The output of Step 10 is a set of prepared input tensors ready for inference by the generative AI model.

### Step 11:

The server executes the generative AI model to generate one or more cooking method candidates.

The input of Step 11 is the set of input tensors from Step 10 and the stored parameters of the generative AI model.

The server performs forward propagation through the layers of the generative AI model by executing matrix multiplications, attention computations, activation functions, and normalization steps on the input tensors.

The server iteratively decodes output tokens by applying a decoding strategy, such as greedy decoding or sampling with top-k or nucleus parameters, using the probability distribution over the vocabulary at each step.

The server concatenates generated tokens to form natural language text that describes one or more recipes, including ingredients and cooking procedures, while respecting the constraints encoded in the extended prompt sentence.

The output of Step 11 is one or more generated text sequences representing cooking method candidates.

### Step 12:

The server parses the generated text sequences into structured result objects.

The input of Step 12 is the generated text from Step 11 and predefined rules or templates that define expected sections and fields.

The server searches for section boundaries, headings, or markers in the text, such as “Title,” “Ingredients,” “Steps,” “Total cost,” and “Total calories,” and divides the text accordingly.

The server extracts lists of ingredients, quantities, and cooking steps as separate lists or arrays, and uses pattern recognition or simple parsing rules to identify numeric cost values and calorie values.

The server constructs structured result objects that contain at least a dish name, a list of ingredients with quantities, an ordered list of cooking steps, an estimated total cost, and an estimated total calorie amount.

The output of Step 12 is a structured representation of each cooking method candidate that can be directly consumed by the terminal.

### Step 13:

The server transmits the structured results to the terminal.

The input of Step 13 is the one or more structured result objects from Step 12 and session information obtained from the request of Step 9.

The server serializes the structured result objects into a transferable format, attaches appropriate identifiers, and sends a response message to the terminal via the communication network.

The server logs summary information, such as processing time and size of generated outputs, for monitoring and optimization.

The output of Step 13 is a network response delivered to the terminal, containing structured recipe data.

### Step 14:

The terminal receives the structured results and prepares display data for the user interface.

The input of Step 14 is the network response from Step 13 and the enriched ingredient dataset from Step 5.

The terminal deserializes the structured result objects, matches ingredient names in the results with entries in the enriched ingredient dataset, and classifies them as owned ingredients or ingredients to be purchased.

The terminal constructs data structures suitable for display, including separate lists for each classification, labels for cost and calorie values, and ordered instruction lists for cooking steps.

The terminal stores the structured results and derived classifications in local memory to support later interactions, such as browsing or saving favorites.

The output of Step 14 is a set of display-ready data structures describing the recipes and ingredient classifications.

### Step 15:

The terminal presents the recipes to the user and optionally updates local stock information.

The input of Step 15 is the display-ready data from Step 14 and user interaction events such as selection of a recipe or confirmation of cooking.

The terminal renders recipe titles, ingredient lists, cooking steps, and numeric indicators (such as total cost and total calories) on the display, and allows the user to scroll, select a recipe, or request additional information.

When the user confirms that a recipe will be used, the terminal deducts estimated quantities of used ingredients from the local stock data by subtracting the recipe quantities from the normalized ingredient quantities, and stores updated stock levels in the local database.

The output of Step 15 is both a visual presentation of the selected recipes to the user and an updated local ingredient stock representation that reflects expected consumption.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

Conventional computer-implemented meal recommendation and ordering systems typically treat recommendation logic, user preference analysis, external menu retrieval, and order processing as separate and loosely coupled components. In many existing architectures, a server retrieves static menu data, applies simple rule-based filtering on price or calories, and returns a list of items, while any personalization or explanation is performed in an ad hoc manner on the client side or is omitted entirely. As a result, such systems suffer from several technical deficiencies.

First, the server-side processing pipeline often fails to integrate heterogeneous data sources—such as user purchase histories, user order histories, inventory information from external systems, and contextual constraints (for example, nutritional limits and budget)—into a unified representation that can be efficiently consumed by advanced inference engines such as generative AI models. The absence of a structured mechanism for transforming multi-source data into machine-readable context causes suboptimal utilization of computational resources, redundant data transfers, and increased latency, because the system either sends excessive raw data to the client or repeatedly queries external services without coordinated filtering.

Second, prior systems usually rely on fixed heuristic rules or manually curated recommendation models, which are rigid and difficult to maintain. These systems are not configured to dynamically construct prompt sentences that encode both numerical constraints (such as calorie limits and budget ranges) and behavioral patterns (such as user preference tendencies) in a form optimized for a generative AI model. Consequently, the recommendation quality is degraded, and the server cannot systematically offload complex reasoning to the generative AI model while preserving control over the input and output formats. This leads to inefficiencies in the use of computing resources on both the server and the external AI infrastructure.

Third, existing architectures tend to separate the recommendation phase from the ordering phase at the protocol and data-structure level. After generating recommendations, the system often discards intermediate linkage information between candidate items, the constraints used for filtering, and the final recommended options. Therefore, when a user selects a recommended item for ordering, the server must perform additional lookups, verification, and re-filtering. This duplication of processing increases server load, complicates database access patterns, and introduces opportunities for inconsistency between what was recommended and what is actually ordered.

Fourth, there is no standardized, server-centric mechanism for constructing and managing machine-readable outputs from generative AI models in the context of meal proposal workflows. Many implementations treat the AI output as unstructured text, which the client must parse or interpret in an ad hoc manner. This leads to brittle client implementations, increased network payload sizes, and difficulty in guaranteeing that the AI output conforms to the constraints required for downstream operations such as order creation and inventory checks.

Accordingly, there is a need for an improved computer-implemented system and server-side processing method that: (i) systematically integrates user-specific historical data, external service data, and contextual constraints into a structured representation; (ii) automatically generates and manages prompt sentences for a generative AI model in a controlled and machine-readable manner; (iii) maintains a coherent linkage between recommendation processing and order processing; and (iv) reduces redundant computations and data transfers, thereby improving overall computational efficiency, scalability, and robustness of meal recommendation and ordering workflows executed by the server.

The specific processing by the specific processing unit 290 of the data processing device 12 in Application Example 1 is realized by the following means.

The present invention provides a server comprising a processor configured to receive, via a user terminal, request information and user identification information and to extract, from the request information, usage conditions including nutritional conditions and cost conditions, to acquire purchase information and order information stored in a storage area based on the user identification information and to generate user characteristics including preference tendencies and cost tendencies by performing statistical processing on the purchase information and the order information, to acquire, via a communication network and based on location information and the usage conditions, meal service information and product service information supplied from an external information processing apparatus and to extract and rank the meal service information and the product service information in accordance with the nutritional conditions, the cost conditions, and inventory conditions, to generate a prompt sentence to be input to a generative AI model based on the user characteristics and the extracted and ranked meal service information and product service information, to supply the prompt sentence to the generative AI model and cause the generative AI model to generate meal proposal information, to specify a plurality of meal candidates that conform to the usage conditions and the user characteristics from the meal proposal information and to transmit the plurality of meal candidates, as structured data, to the user terminal, and to generate order information based on selected meal information received from the user terminal, to transmit the order information to the external information processing apparatus corresponding to the meal service information or the product service information, and to store the order information in the storage area. This enables a technically improved, server-centric processing pipeline in which heterogeneous historical and contextual data are integrated and pre-filtered before being encoded into a structured prompt sentence for a generative AI model, such that the generative AI model produces machine-readable meal proposal information directly aligned with server-maintained constraints, thereby reducing redundant computation, minimizing network overhead, maintaining consistent linkage between recommendation and ordering operations, and improving the overall efficiency, scalability, and robustness of computer-implemented meal recommendation and ordering processes.

The term “processor” refers to a hardware computation unit, or a combination of hardware computation units, configured to execute machine-readable instructions, and may include a central processing unit, a graphics processing unit, a microcontroller, or any other programmable processing circuitry.

The term “terminal” refers to an information processing apparatus operated by a user, including but not limited to a mobile computing device, a stationary computing device, or a display device with input capability, configured to communicate with a server via a communication network.

The term “user” refers to a human operator who interacts with the terminal to input request information, review meal proposal information, and select meal candidates for ordering.

The term “request information” refers to data received from the terminal that indicates at least one condition, preference, or constraint relating to a meal proposal or an order, and may include natural-language text, structured parameters, or both.

The term “user identification information” refers to data used to uniquely identify or associate a user within the system, and may include a user identifier, an account identifier, or equivalent identification data.

The term “usage conditions” refers to conditions extracted from the request information that define constraints or requirements for meal proposals, and may include at least nutritional conditions, cost conditions, and optionally temporal or locational constraints.

The term “nutritional conditions” refers to constraints relating to nutritional properties of meals or products, including but not limited to caloric values, nutrient amounts, or dietary restrictions.

The term “cost conditions” refers to constraints relating to monetary cost, including but not limited to maximum budget, price ranges, or cost preferences for meals or products.

The term “purchase information” refers to data stored in the storage area indicating records of goods or services acquired by the user, including identifiers, prices, timestamps, and associated attributes.

The term “order information” refers to data stored in the storage area indicating records of orders placed by the user for meals or products, including item identifiers, quantities, prices, timestamps, and status information.

The term “storage area” refers to a logical or physical data storage resource accessible by the processor, including but not limited to a non-transitory computer-readable medium, a database, or a memory subsystem.

The term “user characteristics” refers to data derived from purchase information and order information by statistical or computational processing, and representing at least preference tendencies and cost tendencies of the user.

The term “preference tendencies” refers to patterns or profiles indicating the user's favored attributes of meals or products, such as preferred cuisines, ingredients, preparation methods, or tags, inferred from historical usage data.

The term “cost tendencies” refers to patterns or profiles indicating the user's typical or preferred monetary ranges, spending behavior, or cost sensitivity, inferred from historical purchase information and order information.

The term “statistical processing” refers to computational operations performed on purchase information and order information to derive aggregated, summarized, or inferred values, and may include counting, averaging, clustering, ranking, or similar analytic methods.

The term “communication network” refers to a wired or wireless data communication infrastructure that enables data exchange between the server and external information processing apparatuses and between the server and terminals, and may include a local area network, a wide area network, or a public packet-switched network.

The term “location information” refers to data indicating a geographic position or region associated with the user or the terminal, and may include coordinates, an address, or a derived area identifier.

The term “meal service information” refers to data supplied from an external information processing apparatus that describes prepared meals or menu items, including identifiers, descriptions, prices, nutritional attributes, and availability data.

The term “product service information” refers to data supplied from an external information processing apparatus that describes goods usable as ingredients or meal-related products, including identifiers, descriptions, prices, nutritional attributes, and inventory data.

The term “external information processing apparatus” refers to a computing system, separate from the server, that provides meal service information or product service information via the communication network, and may correspond to a provider system for sales facilities or meal-providing facilities.

The term “inventory conditions” refers to conditions relating to the availability or stock status of meals or products, including quantities, stock indicators, or availability flags.

The term “extract and rank” refers to processing operations that select a subset of meal service information or product service information based on one or more conditions, and assign an order or score to the selected information according to at least one criterion.

The term “generative AI model” refers to a machine learning model configured to generate output data, including text or structured data, from input data such as a prompt sentence, and may include a neural-network-based language model or other generative model.

The term “prompt sentence” refers to text or text-like content generated by the processor and supplied as input to the generative AI model, the prompt sentence encoding at least the usage conditions, user characteristics, and candidate meal or product information.

The term “meal proposal information” refers to data generated by the generative AI model in response to the prompt sentence, and representing recommended meals, explanations, or related structured or semi-structured content.

The term “meal candidates” refers to one or more candidate meals or menu items specified by the processor from the meal proposal information, the meal-service information, and the product-service information, and determined to conform to the usage conditions and the user characteristics.

The term “structured data” refers to data arranged in a predefined format or schema, suitable for machine parsing and processing, such as key-value pairs, records, or tabular data.

The term “selected meal information” refers to data indicating one or more meal candidates chosen by the user via the terminal, and may include identifiers, options, quantities, or customization parameters.

The term “sales facility” refers to a physical or virtual establishment that offers products for sale, and that provides product service information to the external information processing apparatus.

The term “meal-providing facility” refers to a physical or virtual establishment that offers prepared meals or menu items, and that provides meal service information to the external information processing apparatus.

The term “candidates” refers to items, including meals or products, that are selected from meal service information and product service information according to at least the cost conditions and nutritional conditions, and that are eligible to be included in the prompt sentence.

The term “cooking plan” refers to proposal information describing preparation methods or combinations of products for producing a meal, derived from candidates included in the prompt sentence and generated by the generative AI model.

The term “order plan” refers to proposal information describing one or more orderable combinations of meals or products, derived from candidates included in the prompt sentence and generated by the generative AI model, and suitable for use in generating order information.

In one embodiment, a server executes a computer program on general-purpose server hardware including one or more central processing units, a main memory, a non-transitory storage device, and a network interface. The server runs an operating system, an application framework, and a database engine. For example, the server may execute a program implemented in a high-level language such as Python, run within a web framework such as a micro web framework, and use a relational database such as a lightweight file-based database system. The server accesses a generative AI model implemented as a large-scale neural network language model, deployed on a separate computation platform or on the same server hardware, via an application programming interface.

The terminal operates as a user-facing client device and may be implemented as a smartphone, tablet, or personal computer. The terminal executes a client application implemented as a native mobile application or as a web application running in a browser. The terminal comprises an input device, such as a touch panel or keyboard, and an output device, such as a display. The terminal is configured to communicate with the server via a communication network using a secure transport protocol.

The user interacts with the terminal by inputting natural-language text, selecting options from graphical user interface elements, and confirming selections. The user provides constraints such as calorie limits, cost limits, preferred food categories, and temporal context, for example, by entering a textual request and adjusting sliders or toggle buttons. The terminal converts the user's input into structured request information that includes both free-text content and explicit numerical or categorical parameters.

The server stores purchase information and order information in the database as structured records. The server represents each record in a normalized schema with fields including user identifiers, item identifiers, timestamps, prices, calorie values, and categorical tags such as cuisine type or preparation method. The server further stores derived user characteristics in separate tables or materialized views, representing preference tendencies and cost tendencies.

The server uses indices on user identifiers and timestamps to enable efficient retrieval and aggregation of historical records.

The server processes usage conditions by mapping them into an internal feature representation. The server converts nutritional conditions such as “under 600 kcal” into numeric features representing upper bounds, and converts cost conditions into numeric ranges. The server also maps categorical preferences such as “prefers Japanese cuisine” into one-hot or multi-hot encoded vectors. The server combines these features with historical features derived from purchase information and order information, such as average price, variance of price, and frequency counts per tag, to form a user feature vector.

The server retrieves meal service information and product service information from one or more external information processing apparatuses. Each external apparatus exposes a programmatic interface for requesting menu data or product data. The server issues requests with query parameters including location information, category filters, and basic nutritional constraints. The server receives structured responses and stores them temporarily in an in-memory data structure, such as a list of records or a table-like structure, each record containing fields such as item identifier, name, price, calories, nutrient breakdown, availability flag, and tag list.

The server performs data processing on the combined internal and external data to extract and rank candidate items. The server applies a multi-stage filtering algorithm. First, the server discards items whose calories exceed the upper limit or whose price exceeds the cost conditions. Second, the server discards items that are marked as unavailable based on inventory conditions. Third, the server computes a similarity score between each remaining item and the user characteristics, using a scoring function that incorporates tag overlap, proximity of item price to the user's typical price range, and alignment with the user's frequent cuisines.

In one embodiment, the server calculates a tag-based similarity component by counting the intersection of item tags and user frequent tags, and normalizing by the size of the union. The server calculates a cost-based similarity component by mapping the difference between the item price and the user's average price into a bounded score. The server calculates a nutritional efficiency component by evaluating a ratio between calories and a normalized nutritional quality score if available. The server combines these components into a total ranking score by applying weighted linear combination. The server then sorts the candidate items by this score, retaining only the top subset.

The server generates a prompt sentence to be supplied to the generative AI model by encoding the usage conditions, the user characteristics, and a summary of the ranked candidate items into a coherent text. The server constructs the prompt sentence using a deterministic template that includes sections for explaining the role of the generative AI model, specifying explicit constraints, summarizing historical user preferences, and listing candidate items in a structured textual format. The server includes an explicit instruction requiring the generative AI model to output data in a predefined machine-readable structure, such as a pseudo-JSON description, but the prompt sentence itself is plain text.

In one concrete example, the user provides the following request via the terminal:

“I want to order a healthy dinner under 600 kcal tonight.”

The server converts this request and the associated user characteristics into a prompt sentence such as:

“You are a diet and menu recommendation assistant using a generative AI model.

The user wants a healthy dinner under 600 kcal tonight.

User constraints:
Maximum calories: 600 kcal
Budget: not specified
Preferred cuisines (inferred): Japanese, Italian

### User Past Order Summary:

2026 Jan. 10: Grilled mackerel set, 580 kcal, 1200 yen, tags: Japanese, grilled, fish
2026 Jan. 15: Chicken salad bowl, 450 kcal, 980 yen, tags: salad, chicken
   Candidate Menus from Nearby Restaurants:
101: Grilled Salmon Set (Healthy Kitchen), 520 kcal, 1300 yen, tags: Japanese, grilled, fish
205: Chicken Salad Plate (Green Garden), 480 kcal, 1100 yen, tags: salad, chicken
332: Tofu & Vegetable Bowl (Veggie House), 410 kcal, 900 yen, tags: vegetarian, low-fat

### Please:

1. Select 3 menus that best match the user's constraints and inferred preferences.
2. Ensure each recommended menu is under 600 kcal.
3. Prefer options that are healthy and budget-friendly.

Return the result as JSON with fields:
“recommended_items”: list of menu IDs
“explanation”: short explanation in Japanese for the user.

Respond only with JSON.”

The server transmits the prompt sentence to the generative AI model via the communication network. In one embodiment, the generative AI model is a transformer-based neural network language model, comprising an embedding layer, multiple self-attention layers, feed-forward layers, and a final projection layer. The model uses multi-head attention with a fixed number of heads and uses positional encodings to capture sequential structure. The model is trained on a large corpus of textual data using an autoregressive objective that minimizes a cross-entropy loss between predicted tokens and ground-truth tokens.

The server uses the generative AI model in an inference-only mode for production workloads. The server forwards the entire prompt sentence as a sequence of token identifiers, generated by a tokenizer, to the model inference engine. The model processes the tokens through successive self-attention and feed-forward computations, using stored weight matrices and non-linear activation functions. The model computes, for each output position, a probability distribution over possible next tokens, and the server controls the decoding process with parameters such as sampling temperature and maximum output length.

The server restricts the output variability and encourages structured output by including explicit formatting constraints and examples in the prompt sentence. The server post-processes the generated text by checking that required keys and structural tokens are present, and by rejecting or correcting outputs that do not satisfy minimal structural rules. The server thereby converts the raw generative output into a validated form that can be directly associated with internal candidate item identifiers.

The server maps the recommended item identifiers and explanation from the generative AI model output back to the previously ranked candidate items. The server then constructs structured data objects containing full details of each recommended item, including names, prices, calories, and tags. The server transmits these objects to the terminal together with the explanation text.

The terminal renders the recommended items in a graphical user interface. The terminal displays each meal candidate with a clear representation of nutritional and cost information and highlights the explanation generated by the generative AI model. The terminal allows the user to select one of the recommended meals and to optionally modify parameters such as portion size or ingredient options.

The server generates order information when the terminal sends selected meal information.

The server links the selected item identifiers to the external meal service information or product service information. The server verifies the current availability and price, and then generates order records in the database with fields such as order identifier, user identifier, item identifiers, final price, and status. The server sends corresponding order commands to the external information processing apparatus. The external apparatus may control physical processes such as kitchen order displays or logistics dispatch systems, thereby connecting the computational process to real-world actions.

The server achieves technical improvements over conventional systems through several mechanisms. The server reduces redundant network traffic by pre-filtering external menu data based on nutritional conditions, cost conditions, and inventory conditions before constructing the prompt sentence, so the server does not send large volumes of raw data to the generative AI model. The server improves processing speed and scalability by using indexed database queries and pre-computed user characteristics, thereby decreasing the computational cost per recommendation request. The server improves accuracy of recommendations by using a controlled prompt sentence that encodes detailed constraints and feature-level preference data, resulting in more consistent and constraint-compliant outputs from the generative AI model.

The server improves data management by employing structured internal representations for historical records, candidate items, and recommendation outputs. By aligning these data structures with the format requested from the generative AI model, the server minimizes transformation overhead and reduces the likelihood of inconsistencies between recommendation and ordering. This structured pipeline also enables efficient logging and replay of specific recommendation scenarios for analysis and debugging, which is difficult in systems that rely on unstructured AI outputs.

The server improves computational efficiency by using a non-conventional processing order: first, performing deterministic filtering and ranking based on numeric and categorical constraints; second, generating a concise prompt sentence containing only high-value candidate items and summarized historical data; and third, delegating only the high-level selection and explanation generation to the generative AI model. This division of labor between deterministic processing and generative inference leads to reduced inference time and reduced token count for the model, which translates to lower latency and lower resource consumption.

The server uses a training approach for the generative AI model that incorporates domain-specific fine-tuning data. In one embodiment, the model is fine-tuned on pairs of structured candidate lists and desired recommendation outputs, using supervised learning with a cross-entropy loss. The server or an associated training system may augment training data by injecting synthetic constraints, such as varying calorie limits and budgets, and by adding perturbations to historical preference patterns. This data augmentation improves the robustness of the model to varied input conditions and enables more stable adherence to constraints. The training process updates model weights using an optimization algorithm such as stochastic gradient descent with adaptive learning rate while monitoring validation performance on task-specific metrics.

The server enforces specific decision rules around the generative AI model's outputs, which differ from typical human decision-making processes. The server checks that every recommended item identifier in the model output corresponds to an item present in the filtered candidate list, and discards any identifier that does not meet this condition. The server then, if necessary, fills missing recommendations by selecting top-ranked items based on the deterministic score. This hybrid rule-based and generative approach ensures that the final output never violates underlying nutritional or cost constraints and that recommendation quality remains high even if the generative AI model output is partially inconsistent.

The server can be configured with multiple alternative embodiments. In one variation, the server uses different generative AI models for different tasks, such as one model tuned for strict constraint adherence and another model tuned for generating richer natural-language explanations. In another variation, the server dynamically adjusts the weight of cost-based or preference-based scores in the ranking algorithm in response to detected user behavior, such as repeated selection of lower-cost items. In yet another variation, the server operates in an offline mode that pre-computes recommended sets for users based on anticipated usage conditions, thereby further reducing response time when the user initiates a session.

The terminal may also adopt multiple implementation variations. In one embodiment, the terminal caches recent recommendation results and allows the user to revisit prior proposals without re-querying the server, thereby reducing network load. In another embodiment, the terminal presents interactive controls for refining conditions, such as a slider for calorie range, and sends incremental updates to the server with lightweight messages, enabling the server to reuse earlier computations and only adjust later stages of the pipeline.

The user benefits from the described system because the server executes a tightly integrated, technically optimized pipeline that a human operator cannot feasibly replicate at comparable speed or scale. The server simultaneously processes large volumes of historical data, external menu data, and multi-dimensional constraints, then systematically structures and compresses this information into a target-specific prompt sentence. This processing, coupled with controlled generative inference and structured output handling, results in improved responsiveness, improved adherence to complex nutritional and budgeting rules, and reduced error rates in recommendation and ordering workflows.

In summary, the server, terminal, and user cooperate such that the server implements a specific, technically grounded architecture for data aggregation, feature extraction, candidate ranking, prompt sentence generation, generative AI model interaction, and order record management. This architecture yields concrete improvements in speed, accuracy, resource utilization, and data consistency relative to conventional systems, and thus constitutes a practical application of generative AI technology and computer processing that goes beyond mere automation of human judgment.

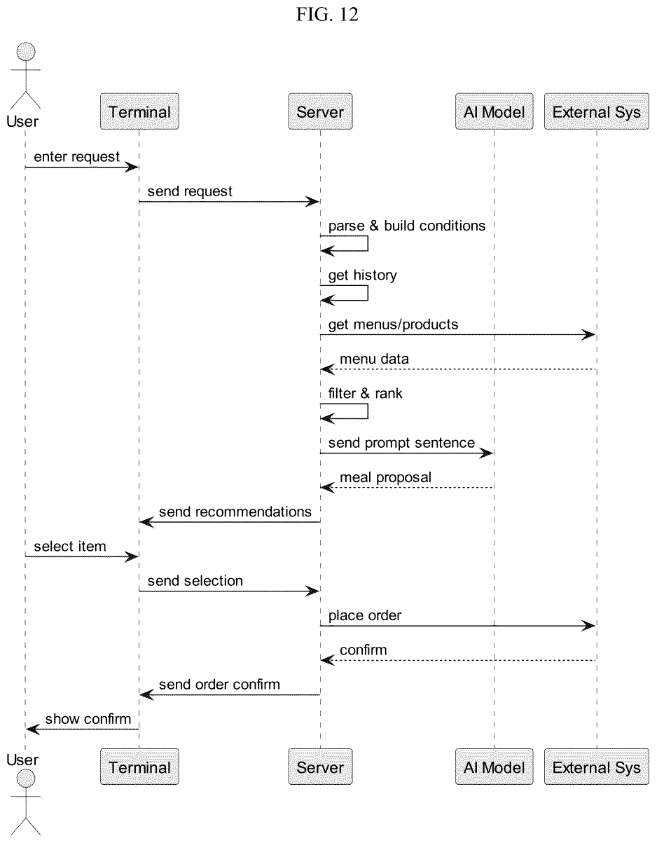

The following describes the processing flow using FIG. 12.

### Step 1:

User operates the terminal to input a meal request and confirm transmission to the server.

User provides input including natural-language text (for example, “I want to order a healthy dinner under 600 kcal tonight.”) and optional explicit parameters such as a budget value or dietary restrictions. Terminal receives this input as raw text and UI control values and converts them into structured request information, including a user identifier, a request text string, numeric values (such as maximum calories and budget), and location information. Terminal performs data formatting by mapping UI elements to key-value pairs and then outputs a structured request object, which terminal transmits to the server via a network connection as the request data.

### Step 2:

Server receives the structured request object from the terminal and parses the contents to identify usage conditions. The input to this step is the request data containing a user identifier, request text, numerical constraints, and location information. Server performs data parsing and validation by checking data types, ranges, and required fields, and then extracts nutritional conditions, cost conditions, and any additional constraints such as preferred cuisine tags from the request text and fields. Server may apply keyword matching or simple rule-based parsing to detect phrases like “under 600 kcal” or “within 10 dollars,” translating them into numeric upper bounds. The output of this step is a normalized usage-condition record that encodes nutritional conditions and cost conditions in machine-usable form, along with the user identifier and location.

### Step 3:

Server retrieves purchase information and order information associated with the user identifier from a storage area. The input to this step is the user identifier and the normalized usage-condition record. Server issues database queries to the storage area to obtain historical records including item identifiers, timestamps, prices, calorie values, and tags. Server performs data aggregation operations such as counting the number of occurrences of each cuisine tag, computing average and median prices, and determining typical calorie ranges of past choices. Server then computes preference tendencies and cost tendencies by applying statistical calculations (for example, frequency distributions and moving averages) to the retrieved records. The output of this step is a user-characteristics record containing numeric and categorical fields that represent preference tendencies and cost tendencies for use in downstream processing.

### Step 4:

Server acquires meal service information and product service information from external information processing apparatuses based on the usage conditions and location information. The input to this step is the usage-condition record, the user-characteristics record, and the location data. Server composes network requests that encode location (for example, coordinates or region codes), basic nutritional limits (such as maximum calories), and category filters, and transmits these requests to external apparatuses providing menu and product data. Server receives structured responses containing lists of items with fields such as name, price, calories, tags, and availability flags. Server then performs initial filtering by discarding items that fail hard constraints, such as exceeding maximum calories or being marked as unavailable. The output of this step is a filtered candidate-item list from external sources, structured in a consistent internal format.

### Step 5:

Server ranks the filtered candidate items according to alignment with user characteristics and usage conditions. The input to this step is the filtered candidate-item list and the user-characteristics record. Server computes a numeric score for each candidate item by applying a ranking function that combines tag overlap, price proximity to the user's typical range, and nutritional efficiency under the given constraints. For example, server calculates a tag similarity value based on the intersection between item tags and frequent user tags, computes a cost deviation value from the user's average price, and evaluates how closely an item's calories fit within the specified nutritional bounds. Server normalizes these components and performs a weighted summation to produce a total ranking score per item. The output of this step is a ranked candidate list, where each item is associated with its computed score and ordering position.

### Step 6:

Server selects a subset of top-ranked candidate items and constructs a prompt sentence for input to the generative AI model. The input to this step is the ranked candidate list, the user-characteristics record, and the usage-condition record. Server selects a predetermined number of top items (for example, the top 20 or top 30) to reduce data volume. Server then generates text segments describing user constraints, historical preferences, and the selected candidate items in a standardized narrative format. Server concatenates these segments into a single prompt sentence that includes explicit instructions to the generative AI model about the number of recommendations, the calorie and cost constraints, and the required output structure. The output of this step is a fully formed prompt sentence encoding all relevant contextual information and constraints in plain text form.

### Step 7:

Server transmits the prompt sentence to the generative AI model and obtains meal proposal information as model output. The input to this step is the prompt sentence generated in the previous step. Server sends the prompt sentence to the generative AI model via an inference interface, which tokenizes the text and processes it through a neural network architecture (for example, a transformer-based language model). Within this architecture, the model performs a sequence of matrix multiplications, attention weight calculations, and non-linear transformations to infer likely next tokens and to construct an output sequence that conforms to the instructions in the prompt sentence. Server collects the model's output text, which describes recommended item identifiers and explanatory content in a machine-readable style. The output of this step is raw meal proposal information in textual form, typically including identifiers of recommended items and associated explanations.

### Step 8:

Server parses the meal proposal information and reconciles it with the ranked candidate list to determine final meal candidates. The input to this step is the raw meal proposal text and the ranked candidate list. Server performs text parsing to extract referenced item identifiers and accompanying explanation text from the model output, and then verifies that each referenced identifier corresponds to an item in the ranked candidate list. Server discards any invalid or unknown identifiers and, if necessary, supplements the set of recommended items with highest-ranking items from the ranked list to reach a target number of recommendations. Server thus applies rule-based validation and correction to align the generative model's proposals with the deterministic constraints. The output of this step is a final recommendation set consisting of validated meal candidates and an explanation string.

### Step 9:

Server formats the final recommendation set as structured data and sends it to the terminal. The input to this step is the final set of recommended meal candidates and the explanation text. Server constructs a structured response that includes for each meal candidate its identifier, name, price, calorie value, and any relevant tags, and adds the explanation text as a separate field. Server may include additional metadata, such as links to images or restaurant information, depending on configuration. Server serializes this structured response into a format suitable for network transmission and sends it to the terminal over the communication network. The output of this step is a recommendation response payload delivered to the terminal.

### Step 10:

Terminal receives the recommendation response payload and displays the meal candidates to the user. The input to this step is the structured response from the server containing recommended meal candidates and explanatory text. Terminal parses the structured data, instantiates internal objects or data structures representing each recommendation, and updates the graphical user interface. Terminal renders each meal candidate with its name, price, calories, and any tags or labels, and presents the explanation text generated based on the generative AI model output. Terminal also enables interaction elements, such as selection buttons, to allow the user to choose one or more recommended items. The output of this step is a visual presentation of the recommendations and an interactive state in which the user can make a selection.

### Step 11:

User inspects the displayed meal candidates and selects one or more items to be ordered. The input to this step is the visual representation of recommended items on the terminal display. User evaluates the presented nutritional, cost, and descriptive information and uses input controls such as taps, clicks, or keyboard actions to select desired items and optional customization parameters (for example, side dishes or portion sizes). Terminal captures the user's selection as selected meal information, including item identifiers and any user-specified options. The output of this step is a structured selection object ready to be sent to the server.

### Step 12:

Terminal transmits the selected meal information to the server for order generation. The input to this step is the structured selection object created from the user's choice. Terminal performs basic validation, such as checking that at least one item has been selected, and then packages the selection together with the user identifier and any payment-related tokens into a structured request. Terminal sends this request to the server via the communication network. The output of this step is an order-request payload delivered to the server.

### Step 13:

Server generates order information, validates availability, and records the order in the storage area. The input to this step is the order-request payload containing user identifier, selected item identifiers, and options. Server cross-references the selected items with the previously obtained meal service information and product service information to verify current availability and correct pricing. Server may perform additional checks, such as re-confirming inventory status through an external apparatus call. Server then computes the final order amount, including taxes and fees, using arithmetic operations on item prices and configured rate parameters. Server creates a new order record in the storage area containing an order identifier, user identifier, item details, total amount, and order status. The output of this step is a confirmed order record and, optionally, a notification to external information processing apparatuses that triggers real-world preparation or delivery processes.

### Step 14:

Server generates an order-confirmation response and sends it to the terminal, and terminal displays the confirmation to the user. The input to this step is the confirmed order record created in the previous step. Server extracts relevant fields such as the order identifier, total amount, and estimated completion or delivery time, and composes a structured confirmation message. Server transmits this message to the terminal via the communication network. Terminal receives the confirmation message, parses its contents, and updates the graphical user interface to show order details and status. User then views the confirmation, which completes the processing flow for the particular request. The output of this step is a displayed confirmation state on the terminal, indicating successful transition from recommendation to finalized order.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

Conventional computer-implemented recipe recommendation systems typically rely on fixed rule engines or simple database filtering. Such systems generally match user inputs against static recipe templates or pre-registered ingredient lists. As a result, these systems suffer from several technical limitations when executed on generic computing hardware. First, conventional systems are not configured to integrate heterogeneous data sources in a coordinated manner at runtime. In particular, typical systems do not systematically aggregate user-specific purchase history data stored in relational databases together with dynamic product data retrieved from external networked information sources, such as store servers providing price, nutrition, and stock information. As a consequence, the processor often performs redundant or suboptimal input/output operations, and cannot construct a coherent, machine-usable representation of the user's actual ingredient possession status and feasible ingredient candidates. This leads to inefficient memory usage, repetitive data transfers, and increased processing latency.

Second, existing systems are not configured to structure the user's natural-language prompt into explicit constraint data that can guide downstream computational processing. For example, textual expressions of target calories, budgets, serving counts, and user preferences are often handled as opaque strings. Without converting such unstructured text into machine-interpretable constraint objects, the processor cannot effectively orchestrate subsequent recipe generation pipelines, resulting in non-deterministic behavior, unpredictable resource consumption, and degraded system responsiveness.

Third, known systems that employ generative AI models commonly pass only the raw user prompt to the model and treat the model output as monolithic text. They do not generate extended prompt sentences that explicitly embed normalized ingredient possession information, ingredient candidate information, and constraint condition data into a single, context-rich input. They also lack mechanisms for validating and iteratively correcting the generative model output. In such architectures, the processor is forced to rely on ad hoc parsing and heuristic post-processing, which increases the rate of malformed outputs, increases the number of failed user interactions, and results in inefficient utilization of computational resources including network bandwidth and external model invocation time.

Fourth, conventional systems do not provide a systematic feedback loop between generated structured recipe data and subsequent prompt sentences. When a user requests a change to previously generated recipes, existing systems typically re-run the entire workflow from scratch, ignoring already computed structured data. This causes repeated database queries, duplicate calls to external services, and unnecessary repeated invocations of the generative AI model, thereby increasing overall processing time and computational load on the server-side processor.

Accordingly, there is a need for an improved computer-implemented system that, when executed by a processor, (i) efficiently integrates multi-source data including purchase history data and external product data, (ii) converts natural-language user prompts into formal constraint condition data, (iii) constructs extended prompt sentences that condition a generative AI model with rich, structured context, (iv) validates and corrects generative outputs through iterative prompt supplementation, and (v) reuses structured recipe data to process subsequent modification requests. Such a system should improve the functioning of the computer itself by reducing redundant data access, improving the determinism and reliability of generative model interactions, and enhancing the efficiency of memory, network, and processor utilization during recipe generation and presentation.

The specific processing by the specific processing unit 290 of the data processing device 12 in Example 2 is realized by the following means.

The present invention provides a server comprising a processor configured to receive user information including a prompt sentence from a user terminal; to identify a user based on the user information and acquire purchase history information associated with the user from a relational database management apparatus; to access an external information providing apparatus based on location information or store identification information included in the user information and to acquire, via a communication network, product information including price information, nutrition information, and stock information of merchandise items; to aggregate the acquired purchase history information to estimate ingredient possession information of the user, normalize the product information to calculate nutrition values and price values per unit amount, and generate ingredient candidate information usable for cooking; to extract, from the prompt sentence, target intake information, budget condition information, number-of-servings information, and preference condition information, and generate constraint condition data; to generate an extended prompt sentence including the prompt sentence, the ingredient possession information, the ingredient candidate information, and the constraint condition data, and to input the extended prompt sentence into a generative information processing model to cause the generative information processing model to generate cooking recipe information satisfying the constraint condition data; to analyze the cooking recipe information output from the generative information processing model and generate structured recipe data including title information, ingredient information, cooking procedure information, nutrition amount information, and cost information; to determine whether the cooking recipe information lacks at least one of title information, ingredient information, and cooking procedure information, and, when the lack is determined, generate an additional input prompt sentence based on the prompt sentence and the structured recipe data, re-input the additional input prompt sentence into the generative information processing model to obtain complemented cooking recipe information, and update the structured recipe data based on the complemented cooking recipe information; and to, when the prompt sentence includes a condition change request for previously generated cooking recipe information, generate a modification request prompt sentence by combining the condition change request with the structured recipe data and input the modification request prompt sentence into the generative information processing model to cause the generative information processing model to generate modified recipe information in which the previously generated cooking recipe information is changed in accordance with the condition change request, and to transmit the structured recipe data including the modified recipe information to the user terminal for visual presentation. This enables the server to more efficiently integrate heterogeneous data sources, to convert natural-language inputs into structured constraint data, to condition a generative AI model with context-rich extended prompt sentences, to iteratively validate and correct generated outputs using already structured data, and to reuse prior structured recipe information for subsequent modification requests, thereby improving the overall computational efficiency, reliability, and responsiveness of the computer-implemented recipe generation system.

The term “user terminal” refers to an electronic device operated by a user, such as a computing device having an input interface, a display interface, and a communication interface, and configured to transmit user information including a prompt sentence to a server and to receive and visually present structured recipe data.

The term “user information” refers to data transmitted from a user terminal to a server, the data including at least a prompt sentence and optionally including user identification information, location information, store identification information, and other context information related to a recipe generation request.

The term “prompt sentence” refers to a natural-language or semi-structured text input provided by a user and transmitted from a user terminal to a server, the text expressing at least one condition or request related to cooking, such as target calories, budget, number of servings, ingredient preferences, or modification requests for previously generated recipes.

The term “relational database management apparatus” refers to a data storage and management system implemented by one or more computing devices configured to store data in relational tables and to execute structured query language commands to retrieve, insert, update, and delete records, including purchase history information associated with a user.

The term “purchase history information” refers to data stored in a relational database and associated with a user, the data including at least identifiers of purchased items, purchase dates, quantities, prices, and optionally store identifiers or categories, and being usable to estimate ingredients likely to be available to the user.

The term “external information providing apparatus” refers to one or more server systems or network-accessible services configured to provide product-related data via a communication network, such as price information, nutrition information, and stock information of merchandise items available at one or more retail locations.

The term “communication network” refers to a wired or wireless data communication infrastructure, such as a local area network, a wide area network, or a public network, enabling data exchange between a user terminal, a server, a relational database management apparatus, and an external information providing apparatus.

The term “product information” refers to data received from an external information providing apparatus and relating to merchandise items, the data including at least price information, nutrition information, and stock information, and optionally including item identifiers, names, categories, and unit sizes.

The term “price information” refers to data indicating a monetary value associated with a merchandise item or a unit amount of the merchandise item, such as a unit price, total price, or price per serving.

The term “nutrition information” refers to data indicating one or more nutritional values associated with a merchandise item or ingredient, such as caloric content, macronutrient content, or micronutrient content per unit amount.

The term “stock information” refers to data indicating availability status of merchandise items at one or more retail locations, such as in-stock, out-of-stock, or estimated quantity available.

The term “ingredient possession information” refers to data representing an estimation of ingredients currently available to a user, the estimation being derived by aggregating purchase history information and optionally applying rules or heuristics, and indicating at least ingredient types and corresponding estimated quantities.

The term “ingredient candidate information” refers to data representing ingredients usable for cooking in a recipe generation process, the data including at least ingredient types derived from product information, normalized unit amounts, associated nutrition values per unit, and associated price values per unit.

The term “normalize the product information” refers to processing product information to convert heterogeneous units, formats, or representations into a unified internal representation, including converting different quantity units into consistent units and calculating nutrition values and price values per unit amount.

The term “target intake information” refers to data extracted from a prompt sentence indicating a numerical or qualitative target related to consumption, such as target caloric intake per serving or per meal.

The term “budget condition information” refers to data extracted from a prompt sentence indicating a monetary constraint, such as a maximum allowable cost per serving, per person, or per meal.

The term “number-of-servings information” refers to data extracted from a prompt sentence indicating the number of persons or servings for which a recipe is to be prepared.

The term “preference condition information” refers to data extracted from a prompt sentence indicating qualitative user preferences or restrictions, such as dietary type, ingredient preferences, ingredient exclusions, seasoning preferences, or cooking time constraints.

The term “constraint condition data” refers to structured data generated by a processor by extracting target intake information, budget condition information, number-of-servings information, and preference condition information from a prompt sentence, the structured data being usable to constrain and guide a recipe generation process.

The term “extended prompt sentence” refers to a text or sequence of tokens generated by a processor and provided as input to a generative information processing model, the text including at least the original prompt sentence, ingredient possession information, ingredient candidate information, and constraint condition data, thereby supplying enriched context for recipe generation.

The term “generative information processing model” refers to a machine-learned model, such as a generative AI model based on a neural network, configured to generate text or structured information in response to an input prompt, and operable on computing hardware separate from or integrated with the server.

The term “cooking recipe information” refers to output data generated by a generative information processing model in response to an extended prompt sentence, the output data including at least textual content describing a recipe, such as a title, ingredients, and cooking procedures, and optionally including nutritional or cost-related descriptions.

The term “structured recipe data” refers to a machine-readable representation generated by a processor by analyzing cooking recipe information, the representation including at least distinct fields or data elements such as title information, ingredient information, cooking procedure information, nutrition amount information, and cost information.

The term “title information” refers to a data element within structured recipe data indicating a name or title assigned to a generated cooking recipe.

The term “ingredient information” refers to one or more data elements within structured recipe data that specify ingredients used in a recipe, including at least item names and associated quantities, and optionally associated nutrition values and price values.

The term “cooking procedure information” refers to one or more data elements within structured recipe data that describe step-by-step instructions for preparing a recipe.

The term “nutrition amount information” refers to one or more data elements within structured recipe data indicating quantitative nutritional values of a recipe or its components, such as total calories per serving.

The term “cost information” refers to one or more data elements within structured recipe data indicating calculated or estimated monetary cost of a recipe or its components, such as total cost per serving.

The term “additional input prompt sentence” refers to a text input generated by a processor based on at least an original prompt sentence and structured recipe data, the text being used as a subsequent input to a generative information processing model for the purpose of complementing or correcting incomplete cooking recipe information.

The term “complemented cooking recipe information” refers to cooking recipe information generated by a generative information processing model in response to an additional input prompt sentence and including content that fills or corrects previously missing or incomplete portions of recipe information.

The term “condition change request” refers to information contained in a prompt sentence indicating a modification of one or more conditions of an already generated recipe, such as changes in dietary constraints, target calories, budget, ingredient preferences, or number of servings.

The term “modification request prompt sentence” refers to a text input generated by a processor by combining a condition change request with structured recipe data corresponding to a previously generated recipe, the text being supplied to a generative information processing model to generate modified recipe information.

The term “modified recipe information” refers to cooking recipe information generated by a generative information processing model in response to a modification request prompt sentence, the information representing a change to previously generated cooking recipe information in accordance with a condition change request.

In one embodiment, a server executes a computer program that implements the claimed system using general-purpose computing hardware and commercially available software components. The server includes at least one central processing unit (CPU), a main memory, a nonvolatile storage device such as a solid-state drive, and a network interface controller. The server executes an operating system, such as a UNIX-like operating system, and a server-side runtime environment, such as a scripting language interpreter or a virtual machine. The server further executes a web application framework to process requests received from user terminals and to coordinate access to a relational database management system and to external information providing apparatuses.

A terminal operates as a user interface device. The terminal includes a display, an input device such as a touchscreen or keyboard, a local processor, and a wireless or wired communication interface. The terminal executes a web browser or a native application. The terminal presents an input field for a prompt sentence, transmits the prompt sentence and associated user information to the server via a network, and renders structured recipe data received from the server on the display.

A user operates the terminal to interact with the system. The user reviews generated recipes displayed by the terminal and may input constraints, preferences, and modification requests as natural-language prompt sentences. For example, the user enters text such as:

“I want to make a dinner of about 600 kcal using what I already have at home.”

“I want to cook dinner for one person with a budget of 250 yen at a nearby supermarket.”

“Using ingredients I often buy, such as chicken, tofu, cabbage, and eggs, please propose a one-week dinner plan under 500 kcal per meal.”

“Based on my purchase history, create a low-salt dinner recipe around 550 kcal that I can cook in less than 20 minutes.”

“Please change the previous recipe to a vegetarian recipe around 600 kcal for two people.”

The server receives such prompt sentences and associated user information, including user identifiers, optional location information, and optional store identifiers. The server uses a relational database management system such as a general-purpose SQL database engine to store and retrieve purchase history information. The server uses foreign key relationships and normalized schemas to represent users, items, purchases, and stores. For example, the database may contain a user table, an item table, a purchase table, and a store table. The server accesses the database through a database client library that issues SQL statements and returns result sets as structured records in memory.

The server uses the purchase history information to estimate ingredient possession information. The server aggregates purchase records for each item by summing quantities and applying decay or consumption heuristics. For example, the server may assign a consumption half-life to perishable items and reduce their estimated availability over time. The server stores intermediate results in in-memory data structures such as hash maps keyed by normalized item identifiers, with values containing estimated remaining quantity and last purchase date. This estimation of ingredient possession information allows the server to construct a closer approximation of the ingredients actually available at the user's location and avoids simply treating all past purchases as available. This contributes to improved accuracy of recipe generation and reduces the need for repeated user input regarding pantry contents.

The server accesses external information providing apparatuses via application programming interfaces exposed over a communication network. Such apparatuses may be retail information servers or nutritional information servers. The server sends HTTP or HTTPS requests including store identifiers, geographic coordinates, or item categories. The external apparatuses return product information encoded in a structured format, such as a list of products including item identifiers, display names, base units (for example, grams, milliliters, pieces), price information, nutritional composition per unit, and current stock status. The server parses the returned data and stores it in memory using indexed collections keyed by item identifiers and store identifiers.

The server normalizes product information to create ingredient candidate information. The server implements unit conversion tables and nutrition conversion functions. When the external apparatus returns heterogeneous units, such as pieces, packs, or grams, the server converts them into a standard unit, for example grams, by applying preconfigured conversion factors stored in tables in the relational database. The server then calculates nutrition values and price values per fixed unit amount, such as per 10 grams or per serving. The server stores the resulting ingredient candidate information in arrays or lists, where each element includes normalized quantity, calories per unit, cost per unit, and an availability flag indicating stock status. This normalization reduces computational complexity in later stages and avoids repeated unit conversions at recipe generation time, thereby improving processing speed and reducing floating-point error accumulation.

The server analyzes each prompt sentence to extract constraint condition data. The server implements a text analysis module that may combine pattern matching with a lightweight natural-language parser. For example, the server uses regular expressions and keyword dictionaries to detect numeric values followed by units such as “kcal,” “calories,” “yen,” “dollars,” or “people.” The server extracts target intake information, such as “600 kcal,” and converts it into a numerical target, such as 600, stored with an associated unit type. The server extracts budget condition information, such as “250 yen per person,” and converts it into monetary constraints per serving. The server extracts number-of-servings information, such as “for one person” or “for two people,” and encodes it as an integer. The server also extracts preference condition information, such as “vegetarian,” “low-salt,” “short cooking time,” or “no dairy,” using keyword lists and simple classification rules. The server aggregates these extracted values into a constraint condition data structure stored as a record with typed fields. By converting unstructured natural-language prompt sentences into explicit constraint condition data, the server improves determinism of subsequent processing and reduces ambiguity in interactions with downstream modules.

The server constructs an extended prompt sentence for a generative AI model. The server combines the original prompt sentence, the estimated ingredient possession information, the ingredient candidate information, and the constraint condition data into a single text sequence. The server uses templates maintained in configuration files to format this information in a consistent manner, for example by grouping sections under headings such as “User Request,” “Available Ingredients,” “Purchasable Ingredients,” and “Constraints.” The server serializes the normalized ingredient data and constraints into human-readable phrases, ensuring that the generative AI model receives explicit numerical values for calories, costs, and servings. The extended prompt sentence forms a structured, context-rich specification that would be difficult and time-consuming for a human to compose manually, particularly given the dynamic combination of database-derived and network-derived data. This improves the quality and predictability of the generative output and reduces the number of iterations required to obtain a satisfactory recipe.

The server interacts with a generative AI model that is implemented as a neural network-based generative information processing model, for example a transformer architecture. The model may be hosted on an external computation service or on dedicated accelerator hardware. The model includes multiple attention layers, feedforward layers, and embedding layers and is trained on large corpora of text data including recipe descriptions and nutritional information. During training, the model minimizes a loss function such as cross-entropy error between predicted tokens and ground-truth tokens by updating weight parameters using gradient-based optimization methods such as stochastic gradient descent or adaptive gradient algorithms. The server does not retrain the model in normal operation but uses an already trained set of weights. The generative AI model tokenizes the extended prompt sentence into subword tokens using a fixed vocabulary, computes contextual representations through stacked attention mechanisms, and generates output tokens sequentially according to probability distributions conditioned on the input and previously generated tokens.

The server instructs the generative AI model to output recipe-related information in a specific structure, for example by including in the extended prompt sentence a requirement that the model produce sections such as “Title,” “Ingredients,” “Steps,” “Total calories,” and “Approximate cost.” The server sets generation parameters such as maximum token length, temperature, and top-k or top-p sampling thresholds to balance diversity and determinism.

Because the extended prompt sentence already includes normalized numeric and categorical information, the model is more likely to produce outputs that meet the specified constraints, reducing the need for redundant calls to the model and improving overall computational efficiency.

The server receives the generated text from the generative AI model and processes it using a parsing module. The server divides the generated text by section headings or markers and uses regular expressions to identify ingredient lines, quantity patterns, nutritional annotations, and cost annotations. The server converts parsed numeric values into internal types such as integers or floating-point numbers and validates them against the constraint condition data. If the server detects missing sections—such as absent title, missing ingredients list, or missing steps—or detects inconsistencies, such as a total calorie amount that deviates beyond a predetermined threshold from the target intake information, the server generates an additional input prompt sentence. The additional input prompt sentence instructs the generative AI model to correct or supplement specific parts of the recipe, for instance “Please add the missing step-by-step instructions” or “Please include an estimated total calorie count per serving.”

The server then provides the additional input prompt sentence, together with selected parts of the previously generated recipe, to the generative AI model. This feedback process forms a controlled loop between structured recipe data and generative outputs. Because the server operates on structured recipe data rather than free text alone, the server can systematically detect and correct model omissions without human intervention. This reduces the number of defective outputs and aligns the model behavior with deterministic system requirements. The iterative correction mechanism decreases network traffic and computational overhead compared to naive repeated generation from scratch, because only constrained portions may be requested for refinement.

When the prompt sentence expresses a condition change request for a previously generated recipe, the server uses the stored structured recipe data as a base. The server combines the condition change request with the existing structured recipe data to create a modification request prompt sentence. For example, the user may input “Change the previous recipe to be vegetarian while keeping about 600 kcal per person.” The server includes the existing ingredients list and steps in the modification request prompt sentence and explicitly indicates which parts should be altered, such as replacing animal-based ingredients with plant-based alternatives and adjusting quantities to maintain the target caloric value. This reuse of structured recipe data prevents the server from recomputing ingredient possession information and ingredient candidate information and avoids repeated external API calls, thus reducing total processing time and network load.

The server stores structured recipe data in a format optimized for retrieval and display, such as serialized objects or normalized tables. The server associates each recipe with metadata, including user identifier, timestamp, underlying constraints, and version information when modifications have been applied. This structure enables the server to reconstruct context for subsequent requests without recomputing earlier stages. The server thereby improves data management within the system, as it uses indexed records and caching strategies to minimize database access latency and improve throughput.

The terminal receives structured recipe data as structured responses from the server. The terminal deserializes the structured data into local objects, arranges the layout of title, ingredients, steps, and nutritional summaries, and renders them on the display with appropriate formatting for readability. The terminal may optionally allow the user to tap or select individual ingredients to view detailed nutritional information or price breakdowns.

The terminal also allows the user to enter new prompt sentences or condition change requests while viewing the recipe, providing an integrated experience.

This architecture provides technical effects beyond simple automation of human tasks. The server employs specific data structures for purchase history information, ingredient possession information, ingredient candidate information, constraint condition data, extended prompt sentences, and structured recipe data. The server uses these data structures to reduce redundant computation, to improve parsing accuracy, and to streamline interaction with the generative AI model. By normalizing units and precomputing per-unit nutrition and cost values, the server reduces the complexity of subsequent calculations and decreases error accumulation. By extracting constraints from natural-language prompt sentences into explicit structures, the server avoids repeated heuristic interpretation and improves the reproducibility of outputs. By integrating dynamic stock information and price information from multiple external sources, the server can generate recipes that are not only nutritionally appropriate but also feasible and efficient in terms of available inventory and budget, which traditional static recipe databases cannot achieve.

From a computational perspective, this system improves processing speed by minimizing unnecessary external calls and redundant database queries through data reuse and caching of structured recipe data. The system improves accuracy by using normalized numeric representations and explicit constraint checking logic to detect inconsistencies in generative outputs. The system improves communication efficiency by structuring the extended prompt sentences in a compact and informative format, which reduces the number of iterative interactions required with the generative AI model and thereby reduces network traffic between the server and the model-hosting hardware.

Additionally, this system uses a generative AI model in a non-conventional manner. Instead of simply delegating all reasoning to the model, the server constrains the model with preprocessed, normalized, and structured context, and then validates and structures the output using deterministic algorithms. The server applies non-standard processing flows, such as iterative prompt supplementation guided by explicit structural checks and modification request prompts built from prior structured data. These flows differ from traditional rule-based recommendation engines and from naive text-generation usage because the server enforces machine-readable structure at both the input and output of the generative AI model.

This tight integration changes how the computing system itself manages and processes data, improving internal resource utilization and control over generative behaviors.

Multiple variations are possible within this framework. The server may implement different strategies for estimating ingredient possession information, such as using probabilistic models based on purchase frequency, household size, or inferred consumption rates. The server may use different generative AI model architectures, such as recurrent neural networks or convolutional sequence models, provided that they perform token-level generation conditioned on an extended prompt sentence. The server may use different relational database schemas or different external information providing apparatuses, including specialized nutritional databases or warehouse management systems. The server may further adjust parsing algorithms, for example using finite-state machines or shallow parsing models, to extract structured recipe data from generative outputs. Each of these variants continues to employ the core concepts of integrating multi-source data, converting natural-language prompt sentences into constraint condition data, constructing extended prompt sentences, validating and correcting generative outputs using structured data, and reusing structured recipe data for subsequent modification requests.

Through these embodiments, the server, terminal, and user cooperate to realize a computer-implemented recipe generation system that is technically distinct from conventional manual recipe selection or simple automated filtering. The system improves internal computer operation by introducing specific data structures, processing flows, and interactions with a generative AI model that yield increased efficiency, improved accuracy, and reduced computational and communication overhead.

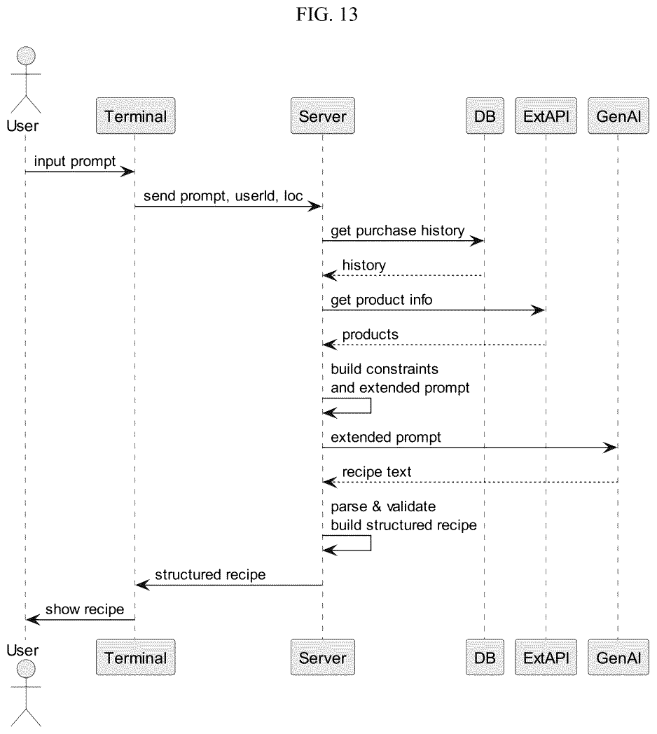

The following describes the processing flow using FIG. 13.

### Step 1:

The user operates the terminal and launches a web browser or a native application.

The terminal displays an input field for a prompt sentence and one or more options for user settings.

Input: User keystrokes or touch input representing a natural-language prompt sentence, optional user login information, and optional location permission.

Processing: The terminal collects the text of the prompt sentence (for example, “I want to make a dinner of about 600 kcal using what I already have at home.”), associates it with a user identifier stored in local memory or obtained from an authentication session, and optionally acquires location information from a location sensor.

Output: A structured request object including the prompt sentence, user identifier, and optional location information, which the terminal transmits to the server via an HTTP or similar network request.

### Step 2:

The server receives the request from the terminal through a network interface.

Input: The structured request object containing the prompt sentence, user identifier, and optional location information.

Processing: The server validates the received data by checking formats (for example, verifying that the prompt sentence is non-empty and that the user identifier is valid). The server parses the request body into in-memory fields, such as ‘prompt_text’, ‘user_id’, and ‘location’. If validation fails, the server constructs an error response; otherwise, it continues processing.

Output: A validated request context in server memory, including the parsed prompt sentence, a confirmed user identifier, and normalized location information.

### Step 3:

The server queries a relational database management system to obtain purchase history information for the identified user.

Input: The user identifier in the validated request context.

Processing: The server constructs one or more SQL queries referencing tables such as a user table, a purchase table, and an item table. The server sends these SQL queries via a database driver to the database engine. The database engine performs index lookups and joins to fetch records representing past purchases. The server receives the result set and converts each row into an internal data structure, for example, objects containing item identifier, item name, quantity, unit, purchase date, price, and store identifier.

Output: A list or array of purchase history records in server memory, organized by item identifier and time.

### Step 4:

The server estimates ingredient possession information from the purchase history.

Input: The list of purchase history records generated in Step 3.

Processing: The server groups the records by item identifier using a hash map or similar associative data structure. For each item, the server sums quantities across purchases and optionally applies decay or consumption rules based on purchase dates and item categories.

The server converts quantities into normalized units (for example, grams or milliliters) using conversion factors stored in the database. The server discards items whose estimated remaining quantity falls below a threshold.

Output: Ingredient possession information, represented as a collection of items with normalized names, estimated available quantities, and last purchase dates.

### Step 5:

The server acquires product information from one or more external information providing apparatuses.

Input: The normalized location information or store identifiers from the request context, and optionally item category filters derived from the prompt sentence.

Processing: The server constructs API requests containing store identifiers, geographic coordinates, or category codes and sends them via HTTP or HTTPS to external servers. External servers perform their own lookups and return product information encoded in structured responses. The server parses these responses, extracting item identifiers, base units, price values, nutritional values per unit, and stock status flags. The server stores this information in in-memory lists indexed by item identifier and store identifier.

Output: A collection of product information records including price information, nutrition information, and stock information for merchandise items available at nearby or specified stores.

### Step 6:

The server normalizes the product information and generates ingredient candidate information.

Input: The collection of product information records from Step 5.

Processing: The server converts diverse units (for example, packs, pieces, and grams) into a standard internal unit using unit conversion tables. For each item, the server computes normalized nutrition values (for example, calories per 100 grams) and normalized price values (for example, cost per 100 grams or per serving). The server also filters out items that are out of stock based on stock information. The server constructs ingredient candidate records containing item name, standard unit, calories per unit, cost per unit, and in-stock indicator.

Output: Ingredient candidate information, represented as a normalized list of candidate ingredients that can be purchased, each with consistent units and associated numeric nutrition and cost values.

### Step 7:

The server extracts constraint condition data from the prompt sentence.

Input: The prompt sentence from the validated request context.

Processing: The server applies text analysis algorithms, including regular expression matching and keyword detection, to identify numeric expressions and associated units (for example, “600 kcal,” “250 yen,” “for one person,” “for two people”). The server maps phrases such as “vegetarian,” “low-salt,” “quick,” or “under 20 minutes” to predefined preference categories. The server stores target intake information as numerical values with units, budget condition information as monetary limits, number-of-servings information as an integer, and preference condition information as a set of flags or labels.

Output: Constraint condition data, encoded as a structured object containing explicit fields for target calories, budget per person, number of servings, and dietary or preparation preferences.

### Step 8:

The server constructs an extended prompt sentence for a generative AI model.

Input: The original prompt sentence, the ingredient possession information from Step 4, the ingredient candidate information from Step 6, and the constraint condition data from Step 7.

Processing: The server formats these inputs into a single textual sequence using template strings or a template engine. The server organizes sections as labeled blocks, such as “User Request,” “Available Ingredients at Home,” “Ingredients Available in Nearby Stores,” and “Constraints.” For each ingredient and candidate, the server inserts human-readable lines listing ingredient name, normalized quantity, calories per unit, and price per unit. The server explicitly describes constraints in words and numbers, such as “Target calories per serving: about 600 kcal” and “Budget per person: up to 250 yen.”

Output: An extended prompt sentence string that encodes all relevant user conditions, available ingredients, and normalized numeric context, ready to be provided to the generative AI model.

### Step 9:

The server invokes the generative AI model with the extended prompt sentence.

Input: The extended prompt sentence from Step 8 and model configuration parameters such as model name, maximum token count, and sampling parameters.

Processing: The server sends a request containing the extended prompt sentence to a model-hosting service or to a local model runtime. The model tokenizes the text into tokens, processes them through layers of a neural network (for example, a transformer with attention mechanisms), and generates output tokens one by one according to learned probability distributions. The server receives the generated token sequence and decodes it into text.

Output: Cooking recipe information in textual form, including sections such as title, ingredients, steps, and optionally calories and costs, as generated by the generative AI model.

### Step 10:

The server parses the generated cooking recipe information into structured recipe data.

Input: The textual cooking recipe information from Step 9.

Processing: The server scans the text for section markers (for example, “Title:”, “Ingredients:”, “Steps:”, “Total estimated calories:”, “Approximate cost:”). The server splits the text into sections and processes the ingredients section line by line, using pattern matching to identify ingredient names, quantities, and any annotated calories or prices. The server processes the steps section as an ordered list of instructions. The server extracts numerical values from the calories and cost sections. The server constructs a structured data object with fields such as title, list of ingredient records, ordered list of steps, total calories, and total cost.

Output: Structured recipe data containing machine-readable title information, ingredient information, cooking procedure information, nutrition amount information, and cost information.

### Step 11:

The server validates the structured recipe data and, if necessary, generates an additional input prompt sentence.

Input: The structured recipe data from Step 10 and the constraint condition data from Step 7.

Processing: The server checks whether mandatory fields (title, ingredients list, steps) are present and non-empty. The server compares the total calories and cost against target intake and budget constraints within tolerance ranges. If the server detects missing sections or significant inconsistencies (for example, total calories deviating beyond a predetermined threshold), the server composes an additional input prompt sentence that instructs the generative AI model to fill in or correct the missing or inconsistent parts. The additional input prompt sentence may include excerpts from the existing recipe and explicit instructions such as “Please add a total calorie count per serving” or “Please provide detailed step-by-step instructions.”

Output: Either validated structured recipe data ready for presentation or an additional input prompt sentence to be used in a further call to the generative AI model.

### Step 12:

The server, when an additional input prompt sentence is generated, refines the recipe by re-invoking the generative AI model.

Input: The additional input prompt sentence from Step 11 and the current structured recipe data.

Processing: The server sends the additional input prompt sentence to the generative AI model, optionally appending relevant parts of the existing recipe as context. The model generates supplementary or corrected text. The server parses this new text in a similar manner to Step 10 and merges the new information into the existing structured recipe data, replacing incomplete or inconsistent sections.

Output: Updated structured recipe data, where missing sections have been filled and inconsistencies relative to the constraint condition data have been reduced or eliminated.

### Step 13:

The server, when the prompt sentence expresses a condition change request for a previous recipe, generates a modification request prompt sentence.

Input: A new prompt sentence containing a condition change request (for example, “Please change this to a vegetarian recipe around 600 kcal for two people.”) and previously stored structured recipe data.

Processing: The server interprets the condition change request using the same text analysis process as in Step 7, producing updated constraint condition data. The server incorporates the existing structured recipe data into a modification request prompt sentence, explicitly indicating which aspects should remain the same and which should change (for example, “Replace animal products with vegetarian ingredients while keeping preparation steps similar and maintaining about 600 kcal per serving.”).

Output: A modification request prompt sentence that combines the condition change request and the prior structured recipe information, ready for input to the generative AI model.

### Step 14:

The server invokes the generative AI model with the modification request prompt sentence to generate a modified recipe.

Input: The modification request prompt sentence from Step 13.

Processing: The server sends the modification request prompt sentence to the generative AI model, receives generated text describing a modified recipe, and parses the text into structured recipe data using the procedure of Step 10. The server may compare the modified recipe against new constraints to ensure compliance.

Output: Modified structured recipe data that reflects changes requested by the user while retaining relevant parts of the original recipe.

### Step 15:

The server transmits structured recipe data to the terminal for presentation.

Input: Validated structured recipe data from Step 10, Step 12, or Step 14.

Processing: The server serializes the structured recipe data into a response format (for example, JSON or another structured representation) and encapsulates it in a network response message. The server sets appropriate status codes and headers and sends the response to the terminal over the network.

Output: A response message containing structured recipe data delivered to the terminal.

### Step 16:

The terminal receives the structured recipe data and displays it to the user.

Input: The response message containing structured recipe data from Step 15.

Processing: The terminal parses the response, constructs local data objects representing title, ingredients, steps, calories, and cost, and renders these on the display. The terminal may format the content in sections, for example, showing the title at the top, followed by an ingredients list with quantities, and then ordered cooking steps and summary of total calories and cost. The terminal may also provide UI controls allowing the user to submit new prompt sentences, including refinement or condition change requests, based on the displayed recipe.

Output: A visual presentation of the recipe on the terminal display, enabling the user to review ingredients, follow cooking procedures, and, if desired, initiate further interactions by entering new prompt sentences.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

Conventional computer-implemented recipe recommendation systems generally employ fixed rule engines or simple filtering over product databases. Such systems typically treat price information, nutrition information, inventory information, and user preference information as independent parameters and combine them by static weighting or hard thresholds. As a result, these systems exhibit several technical limitations.

First, existing systems do not flexibly integrate heterogeneous, time-varying data sources such as user purchase history information, home inventory information, and commodity information of surrounding supply facilities with user emotional state in a unified computational pipeline. In many implementations, emotional or contextual information is either ignored or processed separately from the core recommendation logic, which prevents the underlying computing architecture from exploiting such signals to improve selection and ranking of candidate cooking procedures.

Second, conventional systems do not make effective use of generative AI models in conjunction with structured back-end data and pre- and post-processing logic. In many deployments, a generative AI model is merely given a short natural language prompt sentence without structured incorporation of machine-readable constraints and resource information (for example, budget constraints, nutrition constraints, time constraints, and inventory constraints). This often leads to generated recipes that are inconsistent with database-verified constraints, require unavailable resources, or fail to align with the user's current emotional state. Consequently, the computing resources devoted to model inference are used inefficiently, and the system must perform extensive manual correction or produce results of low utility.

Third, known systems generally lack a closed feedback loop in which user selection results and feedback information are fed back into the computational process to update both prompt-sentence generation and evaluation logic. Without such a loop, the system cannot adapt its internal behavior over time at the level of data flow and processing configuration. This results in a static behavior pattern that does not improve the selection, ordering, and filtering of generated cooking procedure information in response to evolving user behavior and environmental conditions.

Fourth, existing architectures do not provide a standardized mechanism for decomposing the processing flow into: (i) structured acquisition of user input via a terminal, (ii) preprocessing of multi-source resource data by a data processing program, (iii) construction of a constraint-rich natural language prompt sentence, (iv) invocation of a generative AI model as a generative information processing model, and (v) programmatic post-processing and ranking of the model output according to explicit evaluation values. This absence of a structured pipeline leads to ad-hoc integration, difficulty in scaling, and reduced reproducibility and debuggability of system behavior.

Accordingly, there is a need for an improved computer-implemented system that reconfigures how a processor acquires, preprocesses, and fuses user input, purchase history information, inventory information, commodity information, and emotional state; constructs and supplies a constraint-rich prompt sentence to a generative AI model; and post-processes, evaluates, and updates the generated cooking procedure information. Such a system should improve the efficiency and accuracy of the overall information processing, reduce the rate of infeasible or inappropriate recipes, and enhance the adaptability of the system by incorporating user feedback into the core computation path. In other words, the technical problem is to improve the internal operation of the computing system itself—data flows, processing steps, and model interaction—so that generative AI-based recipe generation is more tightly coupled to structured, real-time constraints and to dynamic user state, including emotion.

The specific processing by the specific processing unit 290 of the data processing device 12 in Application Example 2 is realized by the following means.

The present invention provides a server comprising a processor configured to acquire, via a terminal, user input information as structured data, to retrieve from an information storage device purchase history information and inventory information corresponding to the user, to further obtain commodity information of surrounding supply facilities via a communication interface, and to preprocess the purchase history information, the inventory information, and the commodity information by executing a data processing program; to determine, based on the user input information and the preprocessed purchase history information, inventory information, and commodity information, an emotional state of the user by executing an emotion analysis program, and to generate constraint conditions including the emotional state and candidate resource information as a natural language prompt sentence; to transmit input data including the prompt sentence to a generative AI model implemented as a generative information processing model and to cause the generative AI model to perform inference processing so as to generate a plurality of items of cooking procedure information that simultaneously take into account price information, nutrition information, inventory information, and the emotional state; to calculate, for the plurality of items of cooking procedure information, evaluation values based on at least one of a price condition, a nutrition condition, a time condition, and a degree of matching with the emotional state, and to select and rank the plurality of items of cooking procedure information according to the evaluation values; to transmit the selected and ranked cooking procedure information as output data to the terminal to cause the terminal to present the cooking procedure information in a displayable format; and to acquire a selection result and feedback information from the user regarding the cooking procedure information and to update, based on the acquired selection result and feedback information, at least one of processing for generating the prompt sentence and processing for evaluating the cooking procedure information. This enables the computing system to integrate heterogeneous resource data and user emotional state into the construction of a constraint-rich prompt sentence, to control a generative AI model so that its inference output respects database-verified constraints, and to iteratively refine internal processing logic through user feedback, thereby improving the technical performance of the recipe generation pipeline in terms of feasibility, relevance, and computational efficiency.

The term “processor” refers to a hardware information processing apparatus, such as a central processing unit or an execution unit, that executes program instructions to perform the functions described in the present disclosure.

The term “terminal” refers to an information processing device operated by a user, such as a mobile computing device, a handheld computing device, or a client computing device, that provides an interface for inputting information and displaying output information.

The term “user input information” refers to information provided by a user through the terminal, including natural language text, selections, numerical values, or other interaction data indicating requirements, constraints, or preferences.

The term “structured data” refers to data represented in a machine-readable format having an explicit schema, such as a key-value structure, a tabular structure, or a hierarchical structure, including but not limited to formats such as JSON, XML, or relational records.

The term “information storage device” refers to a storage apparatus, such as a memory device or a storage subsystem, configured to store data including purchase history information, inventory information, and commodity information for retrieval by the processor.

The term “purchase history information” refers to records that indicate past acquisitions of resources by the user, including identifiers of acquired resources, quantities, times of acquisition, and other associated attributes.

The term “inventory information” refers to information representing available resources associated with the user, including estimated remaining quantities of previously acquired items or other stock state information for such resources.

The term “commodity information” refers to information describing resources provided by supply facilities, including identifiers, prices, stock quantities, descriptive attributes, and nutrition-related attributes of such resources.

The term “supply facility” refers to a resource-providing entity, such as a commercial establishment or distribution point, that offers commodities which can be acquired by or delivered to the user.

The term “communication interface” refers to a functional unit that enables the processor to exchange data with external devices or systems over a communication network, including sending requests and receiving responses for commodity information.

The term “data processing program” refers to a program executed by the processor to perform operations on data, including but not limited to filtering, aggregating, transforming, joining, or otherwise preprocessing input data into formats suitable for subsequent processing.

The term “emotion analysis program” refers to a program executed by the processor to derive an emotional state of the user from input data such as natural language text, audio data, facial expression data, or explicit emotion labels.

The term “emotional state” refers to a condition representing a psychological or affective status of the user, such as tiredness, happiness, sadness, stress, relaxation, or other similar affective categories.

The term “constraint conditions” refers to limitations or requirements applied to generation or selection of cooking procedure information, including but not limited to conditions related to price, nutrition, time, inventory, and emotional state.

The term “candidate resource information” refers to information describing resources that may be used in a proposed cooking procedure, including resources available in the user's inventory and resources available from supply facilities.

The term “prompt sentence” refers to a natural language instruction sequence, optionally including structured content, that is supplied to a generative AI model to specify a task, constraints, and contextual information for generating output data.

The term “generative AI model” refers to a generative information processing model implemented by machine learning techniques and configured to generate output information, such as text describing cooking procedures, in response to input data including a prompt sentence.

The term “inference processing” refers to processing performed by the generative AI model to compute output information from input information according to parameters learned during a training phase.

The term “cooking procedure information” refers to information describing operations for preparing a meal or dish, including titles, lists of required resources, quantities, and step-by-step instructions.

The term “price information” refers to information indicating a monetary cost associated with resources, including unit prices and total prices computed from quantities of resources.

The term “nutrition information” refers to information indicating nutritional properties of resources or dishes, including but not limited to energy values and composition of nutrients.

The term “time condition” refers to a constraint relating to a temporal aspect of a cooking procedure, including but not limited to preparation time, cooking time, or total required time.

The term “degree of matching with the emotional state” refers to a measure indicating how well a particular cooking procedure information item corresponds to the emotional state of the user, based on criteria such as complexity, style, or associated context.

The term “evaluation value” refers to a numerical or ordered value computed for a cooking procedure information item based on one or more constraint conditions in order to enable comparison, selection, or ranking of multiple items.

The term “select and rank” refers to processing that includes choosing one or more items from a plurality of items based on evaluation values and assigning an order to the chosen items according to the evaluation values.

The term “output data” refers to data generated by the processor and transmitted to the terminal for presentation to the user, including selected and ranked cooking procedure information and associated metadata.

The term “selection result” refers to information indicating which cooking procedure information item or items have been chosen by the user from among the items presented on the terminal.

The term “feedback information” refers to information from the user expressing an assessment or preference with respect to presented cooking procedure information, including but not limited to ratings, comments, or accept-/reject-type indicators.

The term “processing for generating the prompt sentence” refers to operations executed by the processor to construct or modify a prompt sentence based on current and past data, including integration of constraint conditions and candidate resource information.

The term “processing for evaluating the cooking procedure information” refers to operations executed by the processor to compute evaluation values for, and to select or rank, cooking procedure information items generated by the generative AI model.

The term “extended prompt sentence” refers to a prompt sentence obtained by appending or embedding one or more of history information, inventory information, commodity information, and emotional state into an original natural language prompt sentence received from the user.

The term “location information” refers to data indicating a geographical position associated with the user or the terminal, such as coordinates, an address, or a region identifier.

The term “time information” refers to data indicating a temporal point or interval relevant to availability of supply facilities or resources, such as current time, date, or time window.

The term “budget conditions” refers to constraint conditions specifying upper limits or ranges for monetary cost associated with acquisition of resources or execution of cooking procedures.

The term “nutrition conditions” refers to constraint conditions specifying limits or ranges for nutritional characteristics, including caloric value and quantities or ratios of nutrients.

The term “resource information” refers to information describing resources, such as ingredients or items, including identifiers, availability, prices, and nutritional properties, that may be used in a cooking procedure.

The term “resources obtainable within a specific budget” refers to resources that can be acquired while satisfying predetermined budget conditions based on associated price information.

In an embodiment of the invention, the system includes a server and one or more terminals interconnected via a communication network. The server includes at least one processor, a main memory, a non-volatile storage device, and a communication interface. The terminal includes a processor, a display unit, an input unit, at least one sensor (for example, a microphone or a camera), a memory, and a communication interface. The server and the terminal execute programs stored in their respective memories to implement the functions described below.

The terminal executes an application program, such as a native mobile application or a web application running in a browser, that provides a graphical user interface for inputting user requirements. The terminal presents input fields for natural-language text, selectable options for budget, nutrition constraints, preparation time, and emotion icons, and buttons for sending a request. The terminal also optionally acquires audio data from the microphone and image data from the camera. The terminal converts the user input into structured data, for example a key-value representation in memory, and transmits this structured data to the server via the communication interface using a network protocol such as HTTPS.

The server executes an application program implemented, for example, in a general-purpose programming language, and uses a web framework to receive requests from the terminal. The server stores purchase history information, inventory information, commodity information, and configuration data in one or more database management systems such as a relational database management system or a non-relational data store. The server uses a database access library or object-relational mapping library to execute queries and retrieve records.

The server stores purchase history information in a table configured with fields such as user identifier, resource identifier, acquisition quantity, acquisition time, unit price, and nutritional attributes. The server stores inventory information in a table configured with fields such as user identifier, resource identifier, estimated remaining quantity, last update time, and location (for example, “refrigerator”, “pantry”). The server stores commodity information received from supply facilities in one or more tables configured with fields such as facility identifier, resource identifier, unit price, current stock quantity, energy value, protein amount, fat amount, carbohydrate amount, and category identifier.

The server uses a data processing program, implemented for example with numerical computation libraries and tabular data processing libraries, to preprocess the purchase history information, inventory information, and commodity information. The server, for example, loads relevant records into in-memory data structures such as matrices or data frames. The server uses index operations, join operations, and aggregation operations to align resource identifiers and to compute derived values. The server calculates, for example, an estimated current inventory vector for each user by subtracting estimated consumption quantities from historical acquisition quantities according to a consumption model stored in the system. The server also calculates a normalized price vector and a nutrition vector for candidate resources from the commodity information.

The server executes an emotion analysis program to determine an emotional state of the user. In one embodiment, the server processes natural-language text input from the user by applying a text feature extractor. The server converts text into token sequences and then into vector representations, for example by using word embeddings or subword embeddings. The server inputs these feature vectors to a neural network-based classifier configured with multiple layers, such as an input embedding layer, one or more recurrent or transformer layers, and a final classification layer that outputs probabilities for emotion classes such as “tired”, “happy”, “sad”, and “stressed”. The server selects the emotional state corresponding to the maximum probability as the determined emotional state. The server may additionally process audio features, such as mel-frequency cepstral coefficients extracted from voice recordings, using another neural network classifier, and combine the results by weighted averaging to obtain a final emotional state.

The server then generates a prompt sentence as a natural-language instruction for a generative AI model. The server constructs the prompt sentence by combining a task description, constraint conditions, and candidate resource information. The server converts structured constraint data into natural-language segments. For example, the server inserts a budget constraint as a sentence such as “The total cost must be less than 500 yen.” The server inserts nutrition constraints as sentences such as “The total energy must be around 600 kcal and the recipe should be balanced.” The server inserts inventory information as a list of ingredients available at home, and inserts commodity information as a list of ingredients that can be purchased from supply facilities, including price and nutrition values. The server inserts the emotional state as a sentence such as “The user is tired and prefers easy and comforting recipes.”

In one example, the server generates a prompt sentence of the following form:

“Task: You are a cooking assistant. Using the following ingredients already available at home: chicken breast 200 g, carrots 1 piece, onions 1 piece, rice 200 g, and the following ingredients that can be purchased from nearby stores: tofu, spinach, tomatoes, eggs, with their prices and calories as indicated in the list, generate 3 dinner recipes. Constraints: The total cost per recipe must be under 500 yen, the total energy should be around 600 kcal, and the preparation time should be under 30 minutes. The user is tired and wants something easy and comforting. Provide for each recipe a title, a list of ingredients with quantities, and step-by-step instructions.”

In another example, the server generates a prompt sentence:

“Using the following ingredients in the refrigerator: pork 150 g, cabbage ¼ head, miso paste, and the following ingredients that can be bought at nearby stores within a total budget of 250 yen: bean sprouts, tofu, green onions, propose 2 dinner recipes for one person. Each recipe must be less than 600 kcal. The user feels very happy today and wants a special meal for celebration. Include a main dish and a side dish with detailed cooking steps.”

The server transmits the prompt sentence and additional control parameters, such as maximum output length and randomness level, to the generative AI model. The generative AI model is implemented as a generative information processing model, for example as a transformer-based neural network including an embedding layer, multiple self-attention layers, feedforward layers, and a final output layer. The generative AI model has been trained beforehand on large corpora of text including cooking procedures and recipe descriptions. Training uses a loss function such as cross-entropy loss between predicted tokens and ground truth tokens, and employs a weight update algorithm such as stochastic gradient descent or a variant thereof. The model parameters are stored in memory of a computing device with accelerated hardware such as a graphics processing unit or a tensor processing unit.

The server invokes the generative AI model by sending the prompt sentence and receives output text that describes one or more items of cooking procedure information. The generative AI model generates each token sequentially, computing attention weights over previously generated tokens and applying learned weights to predict the next token. This internal structure allows the generative AI model to integrate multiple constraints and contextual segments in the prompt sentence, so that the generated recipes respect budget constraints, nutrition constraints, inventory constraints, and emotional state.

The server performs post-processing on the output text. The server parses the text to segment it into individual recipes, and within each recipe, into a title, ingredient list, and steps. The server maps each ingredient name back to resource identifiers stored in the inventory and commodity tables by using a similarity matching algorithm. The server may use token-based similarity, character-level distance, or a learned embedding similarity to handle variations in naming. The server then recomputes cost and nutrition values for each recipe using the database values for matched resources. The server checks whether each recipe satisfies the budget and nutrition constraints; if not, the server can modify the prompt sentence by adding a clarification such as “The previous recipe exceeded 600 kcal; please adjust the recipe to be under 600 kcal while keeping the same style,” and invoke the generative AI model again.

The server calculates evaluation values for each candidate recipe. The server defines an evaluation function that combines multiple criteria. For example, the server calculates a price score based on the difference between actual cost and target budget, a nutrition score based on the difference between actual calories and target calories and on nutrient balance, a time score based on estimated preparation steps and complexity, and an emotion score based on how well the recipe type and difficulty match the emotional state. The server may represent these scores as numeric values normalized to a common scale. The server then computes a weighted sum or applies another ranking algorithm to produce an overall evaluation value for each recipe. These evaluation operations are implemented in the data processing program using vector operations and matrix multiplications. By using vectorized computations and precomputed indices, the server reduces processing time and enables ranking of many recipes in a short time, which is a technical improvement over manual or rule-based handling.

The server selects a subset of recipes with highest evaluation values and determines an order for presentation to the user. The server packages the selected cooking procedure information, including titles, ingredient lists, quantities, step instructions, cost estimates, nutrition summaries, and explanations of why each recipe was selected (for example, “under 500 yen, around 600 kcal, easy for a tired user”), into structured response data. The server then sends this response data to the terminal via the communication interface.

The terminal receives the response data and converts it into a displayable format. The terminal uses a layout engine to display each recipe as a card or list entry. The terminal may also display icons or labels showing budget, calories, difficulty, and emotion matching. The terminal allows the user to select one of the recipes, to mark recipes as favorites, or to provide feedback such as ratings or comments. The terminal transmits the selection result and feedback information back to the server.

The server stores the selection result and feedback information in the information storage device. The server associates each feedback record with the corresponding prompt sentence, the generated recipes, and the evaluation values used during ranking. The server uses these stored records to adjust both the prompt-generation logic and the evaluation logic. For example, if the user frequently rejects recipes that have long preparation times when the emotional state is “tired”, the server increases the weight of the time score when the emotional state is “tired”. The server may also automatically enrich future prompt sentences with explicit mentions such as “When the user is tired, limit the number of steps and use simple techniques.”

In one embodiment, the server updates parameters of internal recommendation modules that operate before prompt generation. The server may, for example, maintain a preference vector for each user or user cluster, learned using a matrix factorization algorithm or a gradient-boosted ranking model trained on historical selections and feedback. The server uses this preference vector to bias the selection of candidate resources and to adjust training or inference parameters of the generative AI model, such as temperature or output length. This structured data-driven adjustment of prompt composition and evaluation improves technical performance by increasing hit rate and reducing the number of unsatisfactory generations, thereby reducing network calls and computational load on the generative AI model. In another embodiment, the server executes the generative AI model locally as part of the server infrastructure rather than using an external service. The server deploys the model in a container and orchestrates inference requests. The server can adjust model architecture, for example by varying the number of transformer layers, attention heads, or hidden units, to balance latency and accuracy. The server may pre-compute and cache partial encodings for frequently used constraint segments or inventory descriptions, and reuse these encodings in multiple inference calls. This non-conventional use of caching at the level of model inputs reduces processing time and network traffic and is a technical improvement in computing efficiency.

The system architecture produces technical effects that go beyond mere automation of a human recipe selection process. The server arranges data into specific, optimized structures and enforces a particular sequence of preprocessing, prompt-generation, model-inference, and post-processing steps that are tailored to the capabilities and limitations of the generative AI model. By tightly coupling database-verified constraints with generative outputs, the system reduces invalid or infeasible outputs and thus reduces the need for repeated human correction.

The emotion analysis program feeds into the same pipeline, allowing the system to adjust difficulty and style of recipes in a data-driven, algorithmic manner. These interactions improve overall system throughput and accuracy relative to systems where a generative model operates in isolation or with loosely specified prompts.

The system further improves data management by unifying purchase history, inventory, and commodity information in a normalized schema and processing them with vectorized operations. This enables fast filtering and aggregation even when the volume of data is large, such as when a user has many historical purchases and there are numerous supply facilities.

The prompts generated by the server embed compact summaries of this structured data, so that the generative AI model can operate efficiently without directly accessing the raw databases. This partitioning of responsibilities reduces communication load and enables the server to maintain control of data privacy and access.

The invention is not limited to a single type of neural network architecture. The server may employ different generative AI models, such as encoder-decoder transformers, decoder-only transformers, or hybrid architectures, as long as the model accepts a prompt sentence and generates natural-language cooking procedure information. The emotion analysis program may use convolutional neural networks, recurrent neural networks, transformer-based classifiers, or combinations thereof. The ranking and evaluation stage may use simple weighted sums or more complex ranking models, such as learning-to-rank algorithms. The server may adjust loss functions, regularization parameters, and data augmentation techniques during training of internal models to further improve robustness and reduce overfitting.

Alternative embodiments include variations in how the terminal interacts with the server. In some embodiments, the terminal performs part of the emotion analysis, such as speech-to-text conversion or basic sentiment detection, and sends only intermediate features to the server, thereby reducing network bandwidth usage and allowing the server to focus on integration and generation tasks. In other embodiments, the terminal caches recently generated recipes and associated evaluation values, and the server sends only delta updates or ranking adjustments, which further reduces communication load and improves responsiveness.

In yet another embodiment, the system is integrated with control of physical devices such as kitchen appliances. The server or terminal can map specific steps in the cooking procedure information to control commands for appliances, such as setting temperature and duration on a cooking device. The terminal can send such commands via a local network to connected appliances, thus linking the generative AI-based recipe generation with concrete device control. This integration demonstrates that the invention is not limited to abstract data manipulation but extends to technical control of equipment.

Across these embodiments, the server, the terminal, and the user cooperate within a structured, technically oriented pipeline: the server manages complex data processing and model coordination; the terminal handles interaction, input capture, and presentation; and the user supplies requirements, confirms selections, and provides feedback. This arrangement improves processing speed, accuracy of recipe suitability, data management efficiency, and system adaptability, thereby constituting an improvement to computer technology itself rather than an abstract idea or a mere automation of human decision-making.

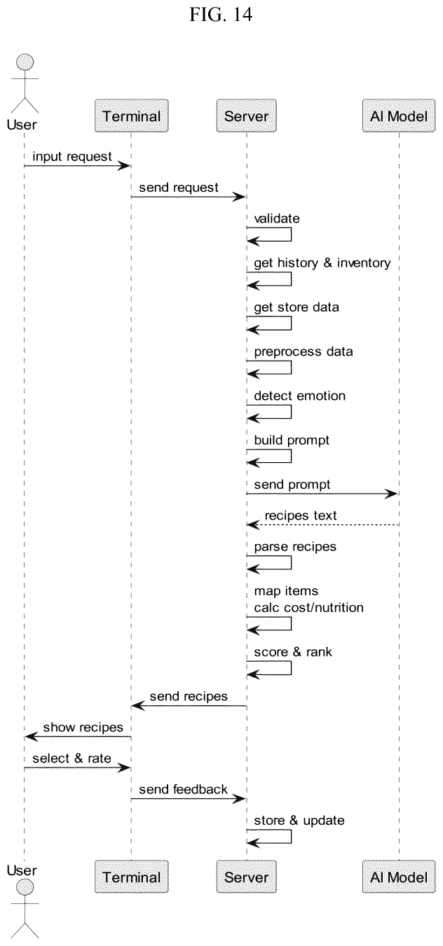

The following describes the processing flow using FIG. 14.

### Step 1:

The user operates the terminal to launch an application and open a recipe request screen.

The input is the user's intent, such as desired ingredients, budget, calories, and current emotion, which the user provides as natural-language text, selectable options, and optionally voice or facial expression.

The terminal displays input fields and controls, receives the text typed by the user (for example, “I want to cook dinner with chicken and vegetables under 500 yen” or “I want a 600 kcal dinner with what I have in my fridge”), captures any selected budget and calorie sliders, and, when enabled, records audio and images via built-in sensors.

The output is a set of raw interaction data elements (text strings, numerical values, selected icons, audio samples, image frames) stored temporarily in the terminal's memory.

### Step 2:

The terminal converts the raw interaction data into structured input data.

The input is the raw text, option selections, and sensor data captured in Step 1.

The terminal tokenizes the text, normalizes character encodings, and maps each field (for example, “budget”, “calorie_limit”, “explicit_emotion”, “free_text”) to corresponding keys in an internal data structure. The terminal compresses or encodes audio and image data if present.

The output is a structured data object that includes user identifiers, location information (if permitted), the natural-language request, numeric constraints, and optional media references, prepared for transmission to the server.

### Step 3:

The terminal transmits the structured input data to the server via a network.

The input is the structured data object created in Step 2.

The terminal opens a secure network connection using a protocol such as HTTPS, serializes the structured data (for example, into a JSON payload), and sends an application-level request to a predefined server endpoint.

The output is a network message delivered to the server's communication interface, containing all user input as a request for recipe generation.

### Step 4:

The server receives the request and performs initial validation.

The input is the network message containing the structured user input data from Step 3.

The server parses the message, verifies the presence and type of required fields (for example, confirms that budget is numeric and non-negative, that calorie limits are within allowed ranges, and that user identifiers are valid), and logs the raw request for auditing. If invalid data is detected, the server constructs an error response.

The output is either a validated request object for further processing or an error response sent back to the terminal.

### Step 5:

The server retrieves purchase history and inventory information for the user.

The input is the validated request object that includes the user identifier.

The server issues retrieval operations against one or more database management systems by forming queries that select purchase history records and inventory records associated with the user. The server then aggregates quantities by resource identifier and time to derive an estimated current inventory state using arithmetic operations (summing acquired quantities and subtracting estimated consumption).

The output is a set of structured records representing the user's purchase history and a derived inventory vector that lists resources and their estimated remaining quantities.

### Step 6:

The server retrieves commodity information from surrounding supply facilities.

The input is the validated request object, including location and time, and internal configuration for accessible supply facilities.

The server identifies candidate supply facilities based on the user's location and current time by comparing location coordinates and operating hours against facility records. The server then calls external or internal interfaces to retrieve commodity information (such as available items, prices, stock quantities, and nutrition attributes), merging responses from multiple facilities into a unified structure.

The output is a normalized commodity information set that contains, for each candidate resource, identifiers, prices, stock states, and nutritional values.

### Step 7:

The server preprocesses purchase, inventory, and commodity information.

The input is the purchase history, the inventory vector, and the commodity information obtained in Steps 5 and 6, together with the constraints in the user request.

The server loads the data into in-memory data structures and performs filtering operations to remove items that are out of stock or that clearly exceed budget or nutrition limits. The server executes joins between inventory and commodity tables by matching resource identifiers and computes derived features, such as per-serving cost and total nutrient values, using vector and matrix operations.

The output is a candidate resource set labeled as “available at home” or “available for purchase”, each with computed cost and nutritional attributes and ready to be used in prompt construction.

### Step 8:

The server determines the user's emotional state by executing an emotion analysis program.

The input is the natural-language request text and any explicit emotion labels or sensor-derived signals included in the validated request.

The server converts the text into tokens and then into numeric feature vectors using a text representation method. The server feeds the vectors into a trained neural network classifier that outputs probabilities over predefined emotion classes. The server optionally processes audio or image features through additional models and combines all outputs through a weighting function to obtain a final emotion label.

The output is an emotional state classification, such as “tired”, “happy”, “sad”, or “stressed”, represented as a label and, optionally, as a probability distribution.

### Step 9:

The server constructs a prompt sentence for a generative AI model.

The input is the preprocessed candidate resource set, the user constraints, and the emotional state derived in Step 8.

The server generates natural-language segments from structured constraints, for example by formatting budget and calorie limits into text and listing available resources with quantities and prices. The server concatenates these segments with a task description and a description of the emotional state to form a coherent instruction, ensuring that the constraints are explicitly specified.

The output is a complete prompt sentence or prompt text that includes task description, resource lists, budget and nutrition constraints, time constraints, and emotional context.

### Step 10:

The server sends the prompt sentence to the generative AI model and obtains generated recipes.

The input is the prompt sentence produced in Step 9 and control parameters such as maximum output length and randomness level.

The server transmits the prompt and parameters to a generative AI model endpoint and initiates inference. The generative AI model internally processes the prompt according to its architecture and generates text that describes one or more cooking procedures. The server waits for completion and captures the returned text.

The output is a generated text body that encodes multiple candidate cooking procedure descriptions resulting from model inference.

### Step 11:

The server parses and structures the generated cooking procedure information.

The input is the generated text body from Step 10.

The server identifies recipe boundaries by detecting markers such as headings or numbering, and within each recipe, extracts titles, lists of ingredients with textual quantities, and step-by-step instructions using pattern recognition and text segmentation logic. The server organizes this extracted information into structured objects for each recipe.

The output is a collection of structured cooking procedure objects, each with a title, ingredient list, quantities, and ordered instructions.

### Step 12:

The server maps recipe ingredients to known resources and recomputes cost and nutrition.

The input is the structured cooking procedure objects and the preprocessed inventory and commodity data from Step 7.

The server performs similarity matching between ingredient names in the recipes and resource identifiers in the stored data, for example by computing distances or scores between text representations and selecting the best match above a threshold. The server then uses the matched resources to calculate total cost and nutrition per recipe by multiplying unit values with required quantities and summing across all ingredients.

The output is an enhanced recipe set in which each recipe includes precise cost and nutrition estimates associated with database-verified resources.

### Step 13:

The server evaluates, selects, and ranks the recipes.

The input is the enhanced recipe set including cost, nutrition, estimated preparation time, and the emotional state.

The server computes evaluation values for each recipe by applying an evaluation function that aggregates a price score, a nutrition score, a time score, and an emotion-matching score, each derived by comparing computed values against target constraints. The server then sorts recipes by their evaluation values and selects a top subset.

The output is a ranked list of selected cooking procedure objects, each associated with an overall evaluation value and, optionally, component scores.

### Step 14:

The server transmits the selected and ranked recipes to the terminal.

The input is the ranked list of selected cooking procedure objects from Step 13.

The server formats the data into an output structure that includes titles, ingredients, quantities, steps, cost and nutrition summaries, and explanatory tags, and sends this structure as a response message through the communication interface to the terminal.

The output is a response message delivered to the terminal that contains all information required for recipe presentation.

### Step 15:

The terminal presents the recipes to the user and collects selection and feedback.

The input is the response message containing the selected and ranked recipes from Step 14.

The terminal parses the message, generates user interface elements for each recipe, and displays titles, images (if any), cost, calorie values, and step-by-step instructions. The terminal then accepts user interactions such as selecting a recipe, saving a favorite, or entering feedback (for example, a rating or comment) and records these interactions.

The output is a selection result identifying one or more chosen recipes and associated feedback information captured on the terminal.

### Step 16:

The terminal sends the selection result and feedback to the server.

The input is the selection result and feedback information collected in Step 15.

The terminal prepares a structured message that includes identifiers of selected recipes, any changes requested by the user, and feedback values, and transmits the message to the server using the same secure communication protocol used for the original request.

The output is a feedback message received by the server, completing the interaction cycle for the current recipe request.

### Step 17:

The server stores the feedback and updates internal processing behavior.

The input is the feedback message containing selection results and user evaluations from Step 16.

The server writes corresponding records into persistent storage, linking each feedback entry to the original prompt sentence, generated recipes, and evaluation values. The server then adjusts internal parameters used for prompt construction and evaluation, such as adjusting weights in the evaluation function or modifying rule thresholds used during preprocessing, according to defined update rules.

The output is an updated configuration and historical record set that modifies how future requests will be processed, thus closing the feedback loop and refining subsequent prompt sentences and recipe evaluations.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

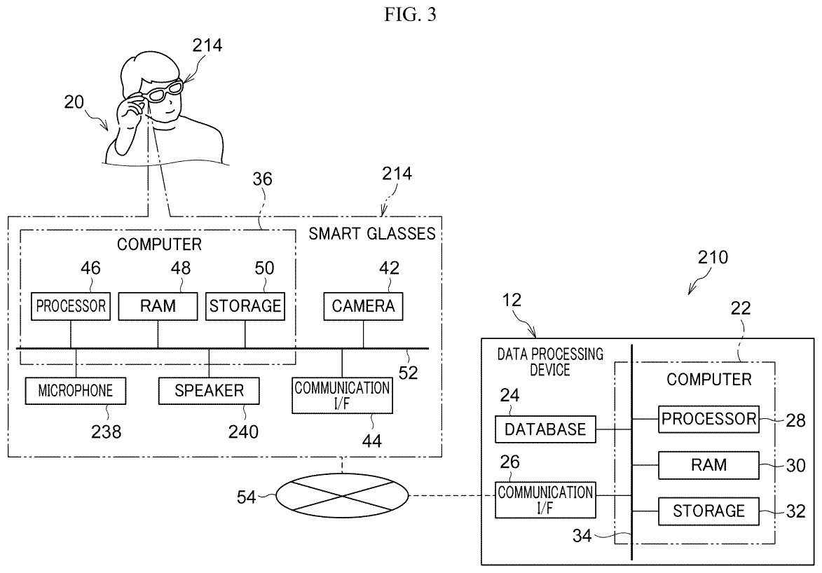

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

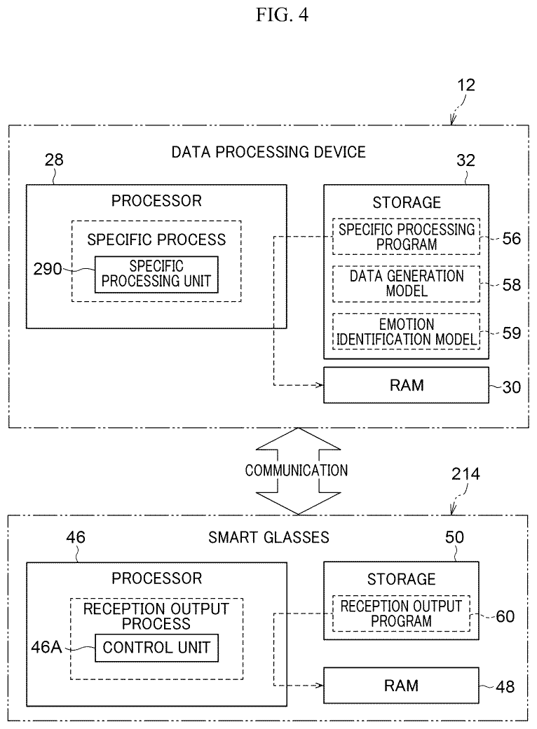

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative Als such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

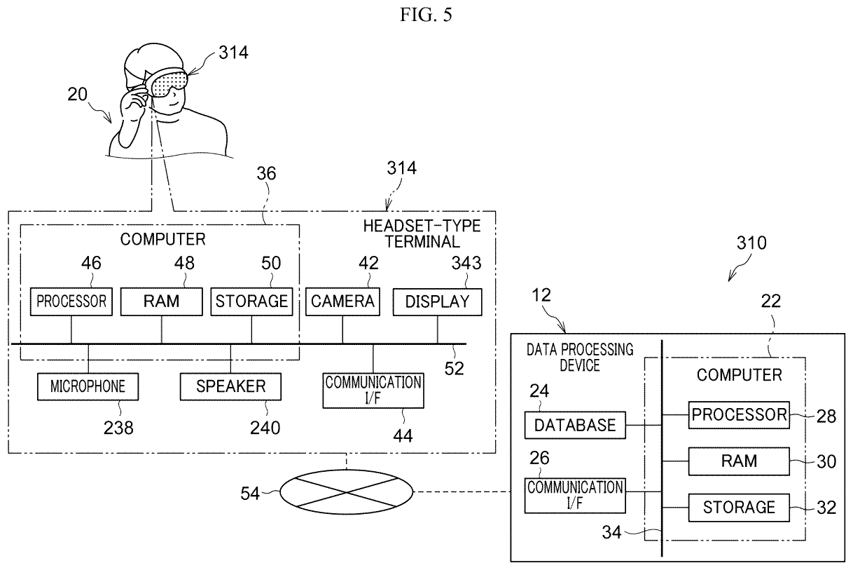

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

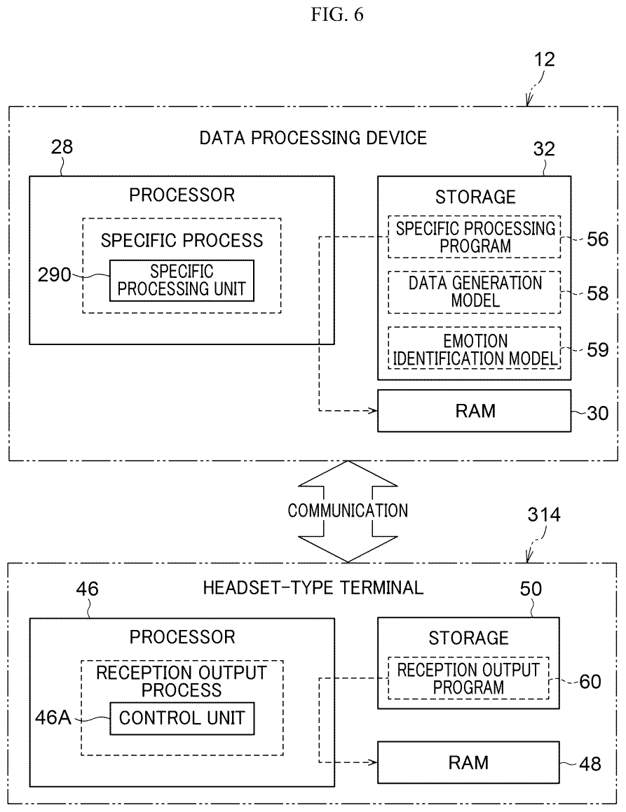

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

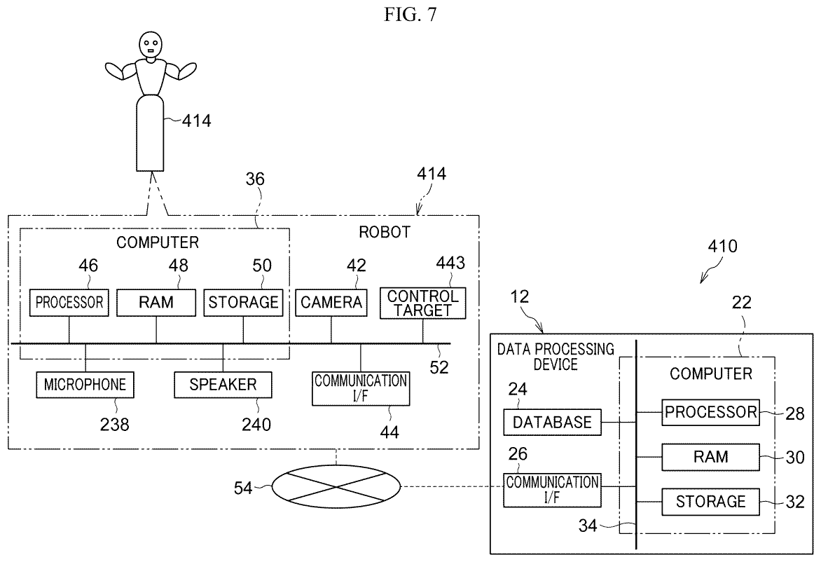

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

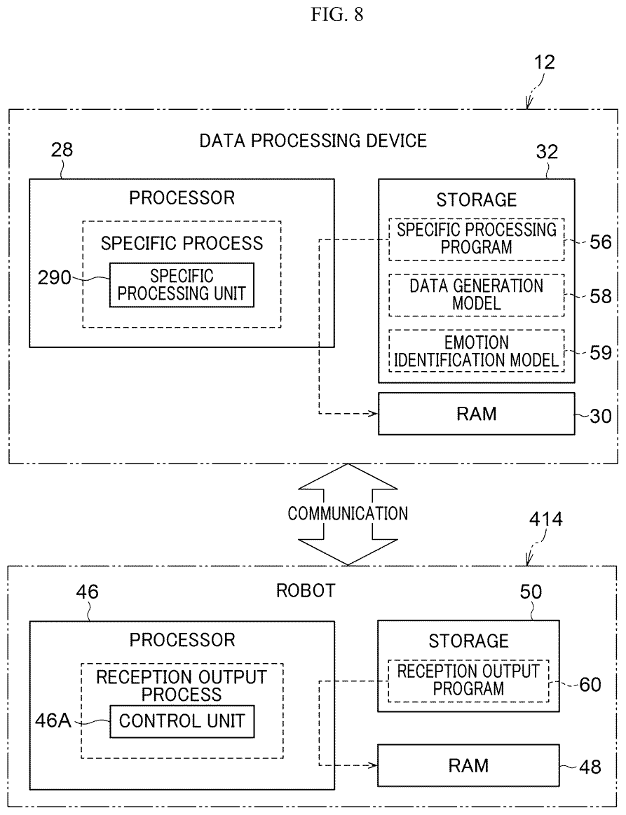

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

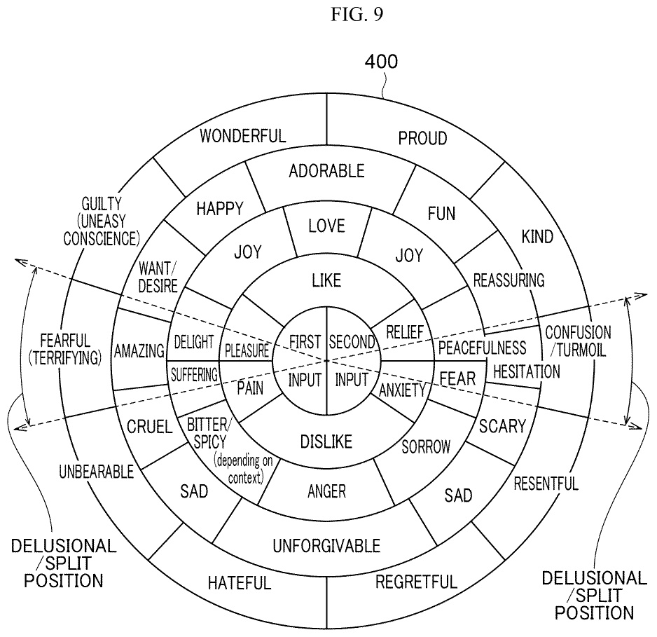

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

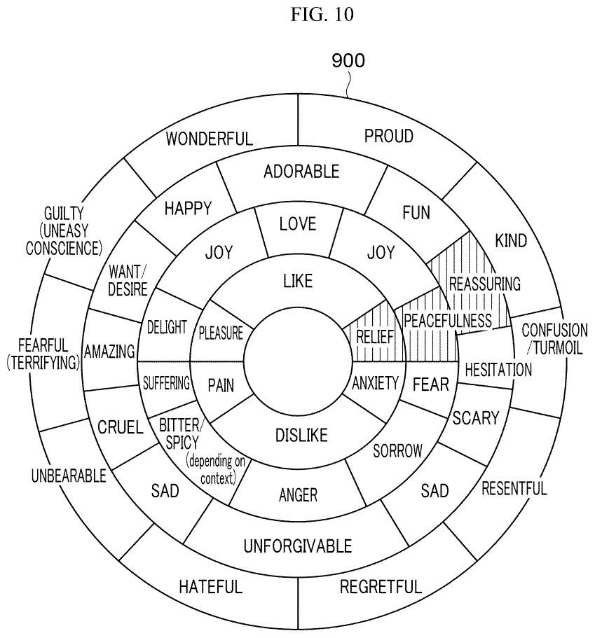

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (Saas).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### Supplementary 1

A system comprising a processor,
wherein the processor is configured to
receive, via an application program executed on an information processing apparatus, a prompt sentence input by a user,
cause the application program to extract, from a storage device of the information processing apparatus in which purchase history data is stored, ingredient information representing food materials currently possessed by the user, and to convert the ingredient information into structured data including attributes related to nutritional values and quantities,
cause the application program to acquire, via a communication network, merchandise information of commercial facilities, the merchandise information including product attribute data comprising at least price information, stock information, and nutritional information, and
to associate the ingredient information with the product attribute data on the basis of product identification information or name information,
cause the application program to perform unit normalization, quantity aggregation, and nutritional value calculation on the ingredient information and the product attribute data, and to generate basic data for specifying usable ingredients and candidate products to be purchased,
cause the application program to analyze the prompt sentence on the basis of predetermined rules or natural language processing and to extract constraint conditions including at least one of a budget condition, a nutritional condition, an intake calorie condition, a serving number condition, and an ingredient usage condition,
cause the application program to construct context information including the constraint conditions, the ingredient information, and the product attribute data as an extended prompt sentence in a hierarchical or segmented text format for input to a generative AI model, and to include specifications of an output format in the extended prompt sentence,
input the extended prompt sentence to the generative AI model and, based on inference processing by the generative AI model, generate one or more cooking method candidates expressed in natural language that satisfy the constraint conditions,
analyze an output from the generative AI model and convert the one or more cooking method candidates into structured results including at least a dish name, a list of ingredients,
quantities, cooking steps, an estimated total cost, and an estimated total calorie amount, and cause the application program to generate a user interface that, on the basis of the structured results, distinguishes between ingredients possessed by the user and ingredients to be purchased, and presents the cooking steps and indicators related to the constraint conditions to the user.

### Supplementary 2

The system according to supplementary 1,
wherein the processor is configured to, when generating the extended prompt sentence, include, as part of the constraint conditions, information specifying a tendency of cooking methods corresponding to an emotional state of the user in addition to a nutritional value condition and a budget condition, and to instruct the generative AI model to output a cooking method candidate that conforms to the emotional state of the user.

### Supplementary 3

wherein the processor is configured to cause the application program to, among the merchandise information of the commercial facilities, extract, from the product attribute data relating to products of a commercial facility specified on the basis of current location information of the user or predetermined region information, combinations of ingredients purchasable within a predetermined budget condition, to include an extraction result in the extended prompt sentence, and thereby to cause the generative AI model to propose a cooking method using ingredients purchasable within the predetermined budget condition.

### Application Example 1

### Supplementary 1

A system comprising a processor,
wherein the processor is configured to
receive, via a terminal operated by a user, request information and user identification information, and extract usage conditions including nutritional conditions and cost conditions from the request information,
acquire purchase information and order information stored in a storage area based on the user identification information, and generate user characteristics including preference tendencies and cost tendencies by performing statistical processing on the purchase information and the order information,
acquire, via a communication network, meal service information and product service information supplied from an external information processing apparatus, based on location information and the usage conditions, and extract and rank the meal service information and the product service information in accordance with the nutritional conditions, the cost conditions, and inventory conditions,
generate a prompt sentence to be input to a generative AI model, based on the user characteristics and the extracted and ranked meal service information and product service information, supply the prompt sentence to the generative AI model, and cause the generative AI model to generate meal proposal information,
specify a plurality of meal candidates that conform to the usage conditions and the user characteristics from the meal proposal information, and transmit the plurality of meal candidates, as structured data, to the terminal, and
generate order information based on selected meal information received from the terminal, transmit the order information to the external information processing apparatus corresponding to the meal service information or the product service information, and store the order information in the storage area.

### Supplementary 2

The system according to supplementary 1,
wherein the processor is configured to
generate the prompt sentence such that, when the request information input by the user includes an instruction to obtain a meal proposal, the prompt sentence includes text information comprising the request information, the user characteristics, and the extracted and ranked meal service information and product service information, and instructs the generative AI model to output the meal proposal information in a machine-readable format.

### Supplementary 3

The system according to supplementary 1,
wherein the processor is configured to
acquire the meal service information and the product service information by obtaining product information provided by a sales facility in proximity to the user and menu information provided by a meal-providing facility, extract candidates that are purchasable within the cost conditions and satisfy the nutritional conditions, and include the candidates in the prompt sentence supplied to the generative AI model so that the generative AI model generates, as the meal proposal information, a cooking plan and an order plan using the candidates.

### Example 2

### Supplementary 1

A system comprising a processor,
wherein the processor is configured to
receive user information including a prompt sentence transmitted from a user terminal; and identify a user on the basis of the user information and acquire purchase history information associated with the user from a relational database management apparatus; and
access an external information providing apparatus on the basis of location information or store identification information included in the user information and acquire, via a communication network, product information including price information, nutrition information, and stock information of merchandise items; and
aggregate the acquired purchase history information to estimate ingredient possession information of the user, normalize the product information to calculate nutrition values and price values per unit amount, and generate ingredient candidate information usable for cooking; and
extract, from the prompt sentence, target intake information, budget condition information, number-of-servings information, and preference condition information, and generate constraint condition data; and
generate an extended prompt sentence including the prompt sentence, the ingredient possession information, the ingredient candidate information, and the constraint condition data, and cause a generative information processing model to generate cooking recipe information satisfying the constraint condition data by inputting the extended prompt sentence into the generative information processing model; and
analyze the cooking recipe information output from the generative information processing model and generate structured recipe data including title information, ingredient information, cooking procedure information, nutrition amount information, and cost information; and transmit the structured recipe data to the user terminal and present the structured recipe data in a format visually displayable on the user terminal.

### Supplementary 2

The system according to supplementary 1,
wherein the processor is configured to
determine that the cooking recipe information output from the generative information processing model lacks at least one of title information, ingredient information, and cooking procedure information, generate an additional input prompt sentence on the basis of the prompt sentence and the structured recipe data, re-input the additional input prompt sentence into the generative information processing model to cause the generative information processing model to regenerate complemented cooking recipe information, and update the structured recipe data on the basis of the complemented cooking recipe information.

### Supplementary 3

The system according to supplementary 1,
wherein the processor is configured to
when the prompt sentence includes a condition change request for previously generated cooking recipe information, generate a modification request prompt sentence by combining the condition change request with the structured recipe data, and input the modification request prompt sentence into the generative information processing model to cause the generative information processing model to generate modified recipe information in which the previously generated cooking recipe information is changed in accordance with the condition change request.

### Application Example 2

### Supplementary 1

A system comprising a processor,
wherein the processor is configured to
receive, via a terminal, input information from a user and acquire the input information as structured data,
analyze the acquired user input information, obtain, from an information storage device that stores purchase history information and inventory information, history information and inventory information corresponding to the user, further obtain, via a communication interface, commodity information of surrounding supply facilities, and perform preprocessing on the history information, the inventory information, and the commodity information by using a data processing program,
determine, based on the user input information and the history information, the inventory information, and the commodity information, an emotional state of the user by using an emotion analysis program, and generate, as a natural language prompt sentence, constraint conditions including the emotional state and candidate resource information,
transmit input data including the prompt sentence to a generative AI model as a generative information processing model, and cause the generative AI model to generate, by inference processing, a plurality of cooking procedure information items that simultaneously take into account price information, nutrition information, inventory information, and the emotional state,
calculate, for the plurality of cooking procedure information items generated by the generative AI model, evaluation values based on at least one of a price condition, a nutrition condition, a time condition, and an emotion-matching degree, and select and rank the plurality of cooking procedure information items according to the evaluation values,
transmit the selected and ranked cooking procedure information items as output data to the terminal and cause the terminal to present the cooking procedure information items in a displayable format, and
acquire a selection result and feedback information from the user regarding the cooking procedure information items, and update at least one of processing for generating the prompt sentence and processing for evaluating the cooking procedure information items based on the acquired result.

### Supplementary 2

The system according to supplementary 1,
wherein the processor is configured to, when a natural language prompt sentence received from the user requests a proposal of cooking procedure information, append the history information, the inventory information, the commodity information, and the emotional state to the prompt sentence to generate an extended prompt sentence, input the extended prompt sentence to the generative AI model, and cause the generative AI model to generate the cooking procedure information.

### Supplementary 3

The system according to supplementary 1,
wherein the processor is configured to, when obtaining the commodity information of the surrounding supply facilities, identify available supply facilities based on location information and time information, extract, from the commodity information of the identified supply facilities, resource information that satisfies predetermined budget conditions and nutrition conditions, and include the resource information in the prompt sentence to be input to the generative AI model, thereby causing the generative AI model to propose cooking procedure information using resources obtainable within a specific budget.

What is claimed is:

1. A system comprising:
a communication interface coupled to a packet-switched network; and
circuitry configured to:
receive, via the communication interface, input data from a terminal device, the input data comprising a request and user identification information;
acquire, from a storage device, history data and attribute data associated with the user identification information;
acquire, via the communication interface, item data from an external data source, the item data comprising attribute parameters including a numerical value parameter, a quantity parameter, and a classification parameter;
perform a normalization operation and an aggregation operation on the history data and the item data to generate constraint data;
generate a prompt sentence based on the input data and the constraint data, the prompt sentence being configured to instruct a generative neural network model to generate structured output data satisfying at least one constraint condition extracted from the input data; and
transmit the structured output data to the terminal device via the communication interface.

2. The system according to claim 1, wherein the circuitry is configured to analyze the input data using a natural language processing operation to extract constraint conditions comprising at least one of a numerical limit condition, a categorical filter condition, and a quantity threshold condition.

3. The system according to claim 2, wherein the circuitry is configured to associate each item in the history data with corresponding item data from the external data source based on an identification parameter or a name parameter, and compute aggregated attribute values for a set of associated items.

4. The system according to claim 3, wherein the prompt sentence comprises a structured template including a constraint field populated with the extracted constraint conditions, a context field populated with at least a portion of the aggregated attribute values, and an output format field specifying a required structure of the structured output data.

5. The system according to claim 4, wherein the structured output data comprises a sequence of procedural steps, each step being associated with at least one item identifier and a quantity parameter, and the circuitry is configured to verify that each item identifier corresponds to an available item in the item data.

6. The system according to claim 5, wherein the circuitry is configured to compute a total numerical value for the structured output data by summing numerical value parameters of all associated items, compare the total with the numerical limit condition, and trigger regeneration of the structured output data when the total exceeds the numerical limit condition.

7. The system according to claim 1, wherein the circuitry is configured to estimate an emotional state of the user based on at least one of text feature data extracted from the input data and interaction pattern data derived from the history data, and incorporate the emotional state into the prompt sentence.

8. The system according to claim 7, wherein the circuitry is configured to adjust a selection criterion in the prompt sentence based on the emotional state such that the generative neural network model generates structured output data reflecting a preference pattern associated with the emotional state.

9. The system according to claim 8, wherein the generative neural network model comprises a transformer architecture having a plurality of self-attention layers, and the circuitry is configured to set inference parameters comprising at least a temperature value and a maximum output token count prior to inputting the prompt sentence.

10. The system according to claim 9, wherein the circuitry is configured to maintain an interaction history in the storage device comprising prior prompt sentences and corresponding structured output data, and incorporate at least a portion of the interaction history into a subsequent prompt sentence.

11. The system according to claim 1, wherein the circuitry is configured to periodically acquire updated item data from the external data source via the communication interface, compare the updated item data with previously stored item data, and update the constraint data based on detected changes in the attribute parameters.

12. The system according to claim 11, wherein the circuitry is configured to compute a scoring value for each candidate item based on a weighted combination of the attribute parameters and a user preference vector derived from the history data, and rank candidate items by the scoring value for inclusion in the prompt sentence.

13. The system according to claim 12, wherein the circuitry is configured to detect an anomaly in the structured output data by computing a deviation metric between expected attribute totals derived from the constraint data and actual attribute totals of the structured output data, and trigger regeneration when the deviation metric exceeds a threshold value.

14. The system according to claim 13, wherein the regeneration comprises modifying the prompt sentence to include an indication of the detected deviation and retransmitting the modified prompt sentence to the generative neural network model.

15. The system according to claim 1, wherein the circuitry is configured to generate a substitution recommendation when an item referenced in the structured output data is determined to be unavailable based on the item data, by identifying an alternative item having similar attribute parameters and updating the structured output data to reference the alternative item.

16. The system according to claim 1, wherein the circuitry is configured to compute, for the structured output data, a plurality of attribute summary values comprising at least a total numerical cost value and a compositional distribution value, and transmit the attribute summary values together with the structured output data to the terminal device.

17. The system according to claim 1, wherein the history data comprises at least one of: transaction records indicating previously acquired items, temporal distribution data indicating acquisition patterns, and preference data derived from user feedback on prior structured output data.

18. A system comprising:
a communication interface coupled to a packet-switched network; and
circuitry configured to:
receive, via the communication interface, input data from a terminal device comprising a request and user identification information;
acquire history data and attribute data from a storage device and item data from an external data source via the communication interface;
perform normalization, aggregation, and association operations on the history data and the item data to generate constraint data;
analyze the input data using a natural language processing operation to extract constraint conditions;
estimate an emotional state of the user based on text feature data extracted from the input data;
generate a prompt sentence based on the constraint conditions, the constraint data, and the emotional state, the prompt sentence being configured to instruct a generative neural network model comprising a transformer architecture;
input the prompt sentence to the generative neural network model and obtain structured output data;
verify that the structured output data satisfies each constraint condition and trigger regeneration when a deviation is detected; and
transmit verified structured output data to the terminal device via the communication interface.

19. The system according to claim 18, wherein the circuitry is configured to train a preference model using a training dataset comprising user interaction records paired with preference labels, the training applying a loss function and updating model parameters using a gradient-based optimization algorithm.

20. A method comprising:
receiving, via a communication interface coupled to a packet-switched network, input data from a terminal device, the input data comprising a request and user identification information;
acquiring, from a storage device, history data and attribute data associated with the user identification information;
acquiring, via the communication interface, item data from an external data source, the item data comprising attribute parameters;
performing a normalization operation and an aggregation operation on the history data and the item data to generate constraint data;
generating a prompt sentence based on the input data and the constraint data, the prompt sentence being configured to instruct a generative neural network model to generate structured output data satisfying at least one constraint condition extracted from the input data; and
transmitting the structured output data to the terminal device via the communication interface.
